(19) United States
(12) Patent Application Publication
(10) Pub. No.: US 2026/0207813 A1
(43) Pub. Date: Jul. 23, 2026

(54) AEROGEL COMPOSITIONS AND METHODS

(71) Applicant: Terasaki Institute for Biomedical Innovation, Los Angeles, CA (US)

(72) Inventors: Yangzhi Zhu, San Diego, CA (US); Reihaneh Haghniaz, Los Angeles, CA (US); Alireza Khademhosseini, Los Angeles, CA (US)

(73) Assignee: Terasaki Institute for Biomedical Innovation, Los Angeles, CA (US)

(21) Appl. No.: 19/140,562

(22) PCT Filed: Dec. 18, 2023
(86) PCT No.: PCT/US2023/084539

§ 371 Date: Jun. 18, 2025

Related U.S. Application Data
(60) Provisional application No. 63/433,630, filed on Dec. 19, 2022.
(60) Provisional application No. 63/433,642, filed on Dec. 19, 2022.
(60) Provisional application No. 63/434,210, filed on Dec. 21, 2022.
(60) Provisional application No. 63/434,214, filed on Dec. 21, 2022.

(51) Int. Cl. A61L 24/00; A61B 5/00; A61B 5/01; A61B 5/259; A61L 24/10

(52) U.S. Cl. CPC A61L 24/0036; A61B 5/01; A61B 5/259; A61B 5/6833; A61L 24/104

(57) ABSTRACT

The present disclosure generally relates to aerogels and methods of producing the same. In some aspects, the disclosure relates to a crosslinked polymer aerogel. The polymer aerogel may comprise a microporous polymeric framework. In some cases, the crosslinked polymeric aerogel comprises gelatin or a derivative thereof. Some aspects of the disclosure further relate one or more physical properties of an aerogel with certain mechanical and/or physical properties (e.g., elasticity, breathability, adhesiveness, etc.). Certain aspects of the disclosure relate to aerogels with an electronic element disposed on a surface of the aerogel. Some aspects relate to methods of producing aerogels such as those described herein.

### RELATED APPLICATIONS

This application claims the benefit of U.S. Provisional Patent Application Ser. No. 63/433,630, filed Dec. 19, 2022, entitled “Aerogel Compositions and Methods;” U.S. Provisional Patent Application Ser. No. 63/434,210, filed Dec. 21, 2022, entitled “Aerogel Compositions and Methods;” U.S. Provisional Patent Application Ser. No.: 63/433,642, filed Dec. 19, 2022, entitled “Flexible Electronics Systems for the Skin and Other Applications;” and U.S. Provisional Patent Application Ser. No. 63/434,214, filed Dec. 21, 2022, entitled “Flexible Electronics Systems for the Skin and Other Applications.” Each of these references is incorporated herein by reference in its entirety.

### GOVERNMENT SUPPORT

This invention was made with government support under AR074234, UG3TR003148, GM126571, GM126831 awarded by the National Institutes of Health. The government has certain rights in the invention.

### FIELD

The present disclosure generally relates to aerogels and methods of producing the same.

### BACKGROUND

Aerogels are a class of synthetic porous materials derived from a gel, in which the liquid component for the gel has been replaced with a gas, without significant collapse of structure. Aerogels have a number of potential industrial applications. However, current aerogels are brittle and undergo catastrophic breakdown under compressive, tensile, shear, and torsional stresses. Aerogels with improved mechanical properties under various stresses would improve the practical application of aerogels. Accordingly, improvements are needed.

### SUMMARY

The subject matter of the present disclosure involves, in some cases, interrelated products, alternative solutions to a particular problem, and/or a plurality of different uses of one or more systems and/or aerogels.

Aspects of the present disclosure relate to an article comprising a crosslinked polymer aerogel comprising gelatin or a derivative thereof, the aerogel having an average pore diameter of less than 50 micrometers, an average pore interconnectivity of at least 65%, and an average moisture permeability of at least 2500 g/(m² day).

Other aspects of the disclosure relate to a crosslinked polymer aerogel comprising gelatin or a derivative thereof. In some embodiments, the aerogel has an average pore diameter of less than 50 micrometers, an average pore interconnectivity of more than 65%, and an average moisture content of less than 1%.

Further aspects of the present disclosure relate to an article comprising a crosslinked polymer aerogel comprising gelatin or a derivative thereof in which the aerogel has an average elastic modulus of between 3 kPa and 6 kPa.

Various aspects of the present disclosure further relate to an article comprising a crosslinked polymer aerogel comprising gelatin or a derivative thereof, the aerogel having an average pore diameter of less than 50 micrometers, an average pore interconnectivity of more than 65%, and an average moisture content of less than 1%.

Additional aspects of the present disclosure relate to articles comprising a crosslinked polymer aerogel comprising gelatin or a derivative thereof, wherein the aerogel has an average elastic modulus of between 3 kPa and 6 kPa.

In another aspect, the present disclosure relates to articles comprising a crosslinked polymer aerogel comprising gelatin or a derivative thereof, the aerogel having an average pore diameter of less than 50 micrometers, an average pore interconnectivity of more than 65%, and an average moisture permeability such that the aerogel swells by less than 1 vol % after absorbing between 500% w/w and 1000% w/w of saline.

In another aspect, the present disclosure relates to articles comprising a crosslinked polymer aerogel comprising gelatin or a derivative thereof, the aerogel having an average pore diameter of less than 50 micrometers, an average pore interconnectivity of less than 65%, and an average moisture permeability such that the aerogel swells by less than 1 vol % after absorbing between 500% w/w and 1000% w/w of saline.

In other aspects, the present disclosure relates to articles comprising a crosslinked polymer aerogel comprising gelatin or a derivative thereof, the aerogel having an average pore diameter of less than 50 micrometers, an average pore interconnectivity of more than 65%, and an average degree of hysteresis of less than 10% when linearly stretched and relaxed by 50% for at least 100 cycles.

In other aspects, the present disclosure relates to articles comprising a crosslinked polymer aerogel comprising gelatin or a derivative thereof, the aerogel having an average pore diameter of less than 50 micrometers, an average pore interconnectivity of less than 65%, and an average degree of hysteresis of less than 10% when linearly stretched and relaxed by 50% for at least 100 cycles.

Additionally, aspects of the present disclosure relate to methods for making an article disclosed herein. For example, in some embodiments, the method comprises crosslinking a polymer comprising gelatin or a derivative thereof in a solution to form a crosslinked solution that causes the crosslinked solution to gel and freeze-drying the gel to form a crosslinked polymer aerogel.

In other aspects, the present disclosure relates methods for adhering any one of the articles disclosed herein to a dermal surface of a subject.

In another aspect, the present disclosure encompasses methods of making one or more of the embodiments described herein, for example, an aerogel. In still another aspect, the present disclosure encompasses methods of using one or more of the embodiments described herein, for example, an aerogel.

Other advantages and novel features of the present disclosure will become apparent from the following detailed description of various non-limiting embodiments of the disclosure when considered in conjunction with the accompanying figures.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates an exemplary process map for the fabrication of one or more aerogels described herein, according to some embodiments;

FIG. 2A illustrates the placement of an exemplary aerogel comprising at least one electronic element disposed on a first surface of the aerogel onto a dermal surface, according to some embodiments;

FIG. 2B illustrates the three-dimensional porous network structure of an exemplary aerogel, according to some embodiments;

FIG. 2C illustrates a top view of the multiple electronic elements disposed on the aerogel surface, including electrochemical, biopotential, temperature, and impedance sensors, according to some embodiments;

FIG. 2D illustrates a flower supporting the weight of an exemplary aerogel, thus indicating the lightweight property of the aerogels, according to some embodiments;

FIG. 2E illustrates a cross-sectional side view of an exemplary aerogel disposed on a dermal surface, according to some embodiments,

FIG. 3A shows a digital image of an exemplary aerogel (left) and a scanning electron microscope (herein “SEM”) image illustrating the micro-scale porous structure of exemplary aerogel, according to some embodiments;

FIG. 3B shows fluorescent optical microscopic images of an exemplary hydrated aerogel stained with Rhodamine-B demonstrating the pore size of said hydrated aerogel, according to some embodiments;

FIG. 3C illustrates the pore size distribution of control aerogels (conventional brittle GelMA) and certain aerogels as discussed herein, according to some embodiments;

FIG. 3D illustrates the interconnected porosity of control aerogels (conventional brittle GelMA) and certain aerogels as discussed herein, according to some embodiments;

FIG. 3E illustrates the tensile strength of control aerogels (conventional brittle GelMA) and certain aerogels as discussed herein, according to some embodiments;

FIG. 3F illustrates the elastic modulus of control aerogels (conventional brittle GelMA) and certain aerogels as discussed herein, according to some embodiments;

FIG. 3G illustrates the tensile strength of control aerogels (conventional brittle GelMA) and certain aerogels as discussed herein, according to some embodiments;

FIG. 3H illustrates the strain failure of control aerogels (conventional brittle GelMA) and certain aerogels as discussed herein, according to some embodiments;

FIG. 3I illustrates the viscoelastic moduli of control aerogels (conventional brittle GelMA) and certain aerogels as discussed herein, according to some embodiments;

FIG. 3J illustrates the cyclic testing of control aerogels (conventional brittle GelMA) and certain aerogels as discussed herein for 1 cycle, 10 cycles, 50 cycles and 100 cycles, according to some embodiments;

FIG. 3K illustrates the moisture permeability of an exemplary aerogel relative to medical tape, polydimethylsiloxane (PDMS), and parafilm, according to some embodiments;

FIG. 3L illustrates the air permeability of an exemplary aerogel relative to medical tape, PDMS, and parafilm, according to some embodiments;

FIG. 3M shows a series of photographs (I-III) illustrating the durability of an exemplary aerogel comprising at least one electronic element disposed on a surface of the aerogel; (I) shows a photograph of an exemplary aerogel with the words “Terasaki Institute” printed with conductive silver ink on a surface of the aerogel connected to an electrical circuit powering a light bulb; (II) shows the aerogel being crushed and crumpled; (III) shows that the crushed and crumpled aerogel retains its conductive ability, according to some embodiments;

FIG. 4A shows microscopic fluorescent images of cells cultured in the incubation medium with exemplary aerogels following treatment with a LIVE/DEAD stain, according to some embodiments;

FIGS. 4B and 4C illustrate quantification of human dermal fibroblast cell viability and fluorescence intensity at 530/590 nm by PrestoBlue assay of FGA after 1, 3, and 7 days of incubation, according to some embodiments;

FIG. 4D illustrates the placement of an exemplary aerogel on a dermal surface of the forearm of a human subject. The aerogel does not induce significant adverse effects after one day of wearing. By contrast, the dermal surface exhibits erythema after 30 min of wearing control gel electrodes, according to some embodiments;

FIG. 4E shows sequential photographs of in vitro biodegradation of exemplary aerogels in phosphate-buffered saline (PBS) solution containing collagenase type II enzyme, according to some embodiments;

FIG. 4F illustrates the weight loss rates of exemplary aerogels within the degradation period of 30 days, according to some embodiments;

FIG. 4G illustrates SEM images to show the surface morphology of exemplary aerogels before and after the 30-day degradation period, according to some embodiments;

FIG. 5A illustrates that the ultraviolet-visible (UV-Vis) reflectance spectrum of exemplary aerogels is between 0.3 and 2.0 micrometers. The normalized ASTM G173 Global solar spectrum is shaded as a reference, according to some embodiments;

FIG. 5B illustrates that the Fourier-transform infrared (FTIR) spectrum of exemplary aerogels is between 5 and 17 micrometers. The human-body mid-IR radiation is shaded as a reference, according to some embodiments;

FIG. 5C illustrates a photographic image of an exemplary aerogel attached to a dermal surface of a human subject. The inset shows SEM micrographs illustrating the porous structure of the aerogel, according to some embodiments;

FIG. 5D illustrates thermal mapping of an exemplary aerogel on a dermal surface of a human subject, recorded using an infrared (IR) thermal camera, according to some embodiments;

FIG. 6A shows a graphic representing on-body validation experiments using exemplary aerogels to monitor glucose and alcohol levels, according to some embodiments;

FIG. 6B shows (I-II) electrochemical sensor signal recordings for interstitial fluid (ISF) glucose before and after food intake; (III) comparison between the glucose levels in ISF measured using the electrochemical sensor and in blood using a blood glucose meter, according to some embodiments;

FIG. 6C shows (IV-V) electrochemical sensor signal recordings for sweat alcohol levels before and after wine intake; (VI) comparison between the alcohol levels in sweat measured using the aerogels and in blood using a commercial blood-lactate meter, according to some embodiments;

FIG. 7A shows a graphic of a human subject using exemplary aerogels for multiplexed chemical-electrophysiological analysis during exercise, according to some embodiments;

FIG. 7B shows (I-II) electrochemical sensor signal recordings for ISF glucose levels before and after high-intensity exercise; and (III) comparison between the glucose levels in ISF measured using the aerogels and in blood using a commercial blood-glucose meter, according to some embodiments;

FIG. 7C shows (IV-V) electrochemical sensor recordings for sweat lactate levels before and after exercise; and (VI) comparison between the alcohol levels in sweat using the aerogels and blood using a commercial blood-alcohol meter, according to some embodiments;

FIG. 7D shows (VII-VIII) physiological signal recordings for skin temperature variations before and after exercise; and (IX) comparison between skin temperature measured using the FGA-based E-skin and a commercial infrared thermometer, according to some embodiments;

FIG. 7E shows (X-XI) physiological signal recordings for skin impedance/hydration levels before and after exercise and (XII) comparison between the skin impedance/hydration levels measured using the aerogels and commercial hydration sensor, according to some embodiments;

FIG. 7F shows (XIII-XIV) physiological signal recordings for electrocardiogram (ECG) before and after exercise. (XV) Comparison between the heart rate measured using exemplary aerogels and commercial ECG monitor.

FIG. 8A shows the continuous signal recording showing the sweat-lactate profile during high-intensity stationary cycling. Dashed lines mark the time corresponding to the plotted physiological signals recordings before, during, and after exercise, according to some embodiments;

FIG. 8B shows the validation of an exemplary aerogel using a commercial blood-lactate meter. Multiplexed signal readings before, during, and after stationary cycling, according to some embodiments;

FIG. 8C shows the comparison between skin hydration signal measured by a commercial skin hydration level and exemplary aerogels before, during, and after cycling, according to some embodiments;

FIG. 8D shows a comparison between skin temperature signal measured by a commercial thermometer and an exemplary aerogel before, during, and after cycling, according to some embodiments;

FIG. 8E shows a comparison between heart rate signal measured by a commercial electrocardiogram (ECG) monitor and an exemplary aerogel before, during, and after cycling, according to some embodiments;

FIG. 9 illustrates a hypothetical array of electrical elements for electrical stimulation of a Vagus nerve of a subject, according to some embodiments.

FIG. 10 shows a digital image of a fabricated aerogel comprising at least one electrical element on its surface fabricated using a scalable screen-printed technique, according to some embodiments;

FIG. 11 shows magnified photomicrographs of each assigned sensor/core component from the aerogel comprising at least one electronic element. The magnified regions highlight the sensor components and other interconnections, including electrochemical, temperature, hydration, and biopotential sensor, serpentine interconnections, and flexible flat cable, according to some embodiments;

FIG. 12 illustrates the mechanism of the impedance sensor based on interdigitated electrodes, according to some embodiments;

FIG. 13 illustrates the impedance-frequency curve of exemplary aerogels characterized under various hydration states as determined by a commercial hydration meter.

FIG. 14 illustrates the in vitro validation of the impedance monitoring of an exemplary aerogel comprising at least one electrical element, according to some embodiments;

FIG. 15 illustrates the thermoelectric behavior of an exemplary aerogel, according to some embodiments;

FIG. 16 illustrates the temperature recorded by an exemplary aerogel comprising an electronic element during the process of heating and the subsequent cooling;

FIG. 17 illustrates a continuous ECG signal acquired using an exemplary aerogel comprising at least one electronic element, according to some embodiments;

FIG. 18 shows high-quality ECG signals detected from a subject wearing an exemplary aerogel comprising at least one electronic element when the subject is at rest, according to some embodiments;

FIG. 19 shows no signal detected by aerogels comprising at least one electronic element without the surface modifications of glucose oxidase (GOx) before and after the meals, according to some embodiments;

FIG. 20 shows no signal was detected by aerogels comprising at least one electronic element without the surface modifications of alcohol oxidase (AOx) after the alcohol-intake, according to some embodiments;

FIG. 21 shows that no signal was detected by aerogels comprising at least one electronic element without the surface modifications of lactate oxidase (LOx) after the exercise, according to some embodiments;

FIG. 22 shows the amperometric response of an exemplary aerogels comprising at least one electronic element to successive additions of 2 mM glucose from 0 to 10 mM, according to some embodiments;

FIG. 23 shows the glucose selectivity of exemplary aerogels comprising at least one electronic element in the presence of glucose (Glu, 2 mM), lactate (LAC, 10 mM), ascorbic acid (AA, 10 micromolar), uric acid (UA, 10 micromolar) PBS, according to some embodiments;

FIG. 24 shows the amperometric response of exemplary aerogels comprising at least one electronic element to successive additions of 20 mM alcohol from 0 to 100 mM, according to some embodiments;

FIG. 25 shows the alcohol selectivity of exemplary aerogels comprising at least one electronic element in the presence of ethanol (Eth, 20 mM, 40 mM), glucose (Glu, 1 mM), ascorbic acid (AA, 10 micromolar), uric acid (UA, 10 micromolar), and PBS, according to some embodiments;

FIG. 26 shows the amperometric response exemplary aerogels comprising at least one electronic element to successive additions of 5 mM lactate from 0 to 20 mM, according to some embodiments;

FIG. 27 shows the lactate selectivity of exemplary aerogels comprising at least one electronic element in the presence of lactate (Lac, 2 mM, 4 mM), glucose (Glu, 1 mM), ascorbic acid (AA, 10 micromolar), uric acid (UA, 10 micromolar) and PBS, according to some embodiments;

FIG. 28 shows an illustration of different ice crystal formations during aerogel fabrication. Ice crystals formed under liquid nitrogen regime before and during polymerization of GelMA are smaller and highly connected, leading to the flexibility of the aerogels, according to some embodiments;

FIG. 29 shows the structure stability of exemplary aerogels of the present invention during folding and rolling, according to some embodiments;

FIG. 30 shows a SEM photomicrograph of the conventional brittle GelMA aerogel (BGA) to show its porous structure, according to some embodiments;

FIG. 31 illustrates the pore volume of exemplary aerogels of the present invention, according to some embodiments;

FIG. 32 shows a comparison of water absorption between exemplary aerogels of the present invention and control aerogels, according to some embodiments;

FIG. 33 shows a comparison of moisture transport between exemplary aerogels of the present invention, medical tape, PDMS, and Parafilm.

FIG. 34 shows water contact angle of exemplary aerogels disclosed herein (top) before and (bottom) after hydration, according to some embodiments; and

FIG. 35 shows changes in relative resistance of exemplary aerogels comprising at least one electronic element under 100 cycles of bending (left) and twisting (right) deformation, according to some embodiments;

FIG. 36A (left) shows a schematic illustration of the wound closure experimental setup in which the skin was stretched by the external force. (Right) shows representative force-displacement curves obtained from the wound closure experiments, according to some embodiments;

FIG. 36B (left) shows photographs of an exemplary aerogel disposed on a dermal surface of a porcine subject before (i) and after (ii) applying an external force. The external force delaminated the aerogel from the dermal surface without rupturing the aerogel; (right) shows the adhesive strength of an exemplary aerogel after different incubation times on a dermal surface of a porcine subject, according to some embodiments;

FIG. 37 shows on-body conformability and mechanical integrity of exemplary aerogels comprising at least one electronic element during twisting, bending, and after these deformations, according to some embodiments; and

FIG. 38 shows FTIR spectra of the flexible GelMA aerogel and;

FIG. 39 shows continuous monitoring of the skin impedance using an exemplary aerogel comprising at least one electronic element, according to some embodiments.

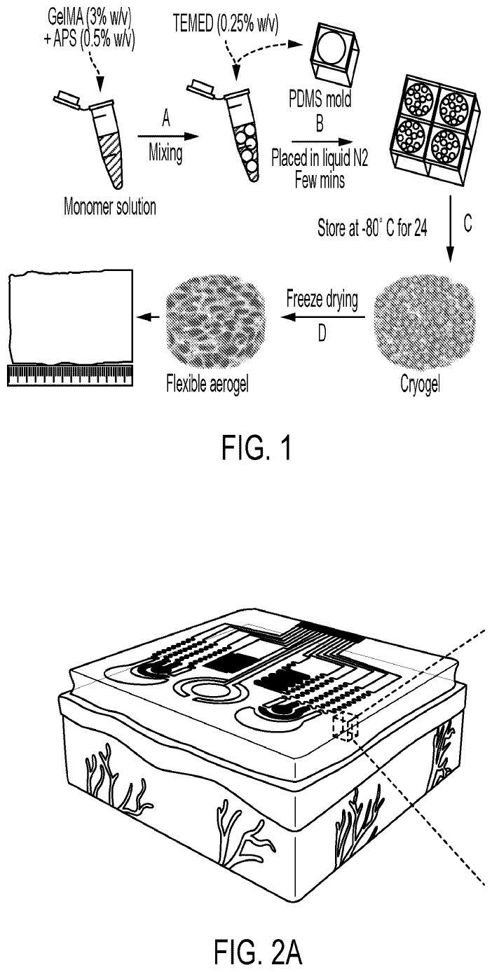

### DETAILED DESCRIPTION

The present disclosure generally relates to aerogels and methods of producing the same. In some aspects, the disclosure relates to a crosslinked polymer aerogel. The polymer aerogel may comprise a microporous polymeric framework. In some cases, the crosslinked polymeric aerogel comprises gelatin. Some aspects of the disclosure further relate to one or more physical properties (e.g., pore size, density, etc.) of an aerogel that impart the aerogel with one or more desired functional properties (e.g., elasticity, adhesiveness, etc.). Certain aspects of the disclosure relate to aerogels with an electronic element disposed on a surface of the aerogel. Other aspects relate to methods of producing aerogels such as those described herein.

Certain embodiments are generally directed to aerogels that are unexpectedly flexible. Such aerogels may be used in a variety of different applications, including as a material that can be adhered to the skin of a subject over multiple days, and in some cases, without using tape or other external devices. The aerogels may be formed from materials such as gelatin, for example, as gelatin methacryloyl and/or other crosslinked gelatin formulations. Such gelatins may be particularly useful, e.g., due to their biocompatibility and ease of preparation. The aerogel may also include one or more electrical elements in certain embodiments, for example, for stimulating nerves (e.g., the vagus nerve) within a subject, and/or for determining nerves or other electrical activity within the subject.

Without wishing to be bound by any theory, it is believed that certain aerogels such as those discussed herein may be flexible due to their pore distribution and interconnectivity. Such physical properties can be achieved, for example, by quickly freezing a monomer solution to form the aerogel, e.g., in liquid nitrogen. As discussed herein, under such conditions, relatively small pores may form under such conditions, allowing the resultant aerogel to exhibit a surprising amount of flexibility. In contrast, many prior art aerogels have larger pores and/or smaller pore interconnectivity, and exhibit brittleness and less flexibility. Such aerogels are not useful for adhesion to the skin of a subject, as movement of the subject will break the aerogels and cause them to fall off; accordingly, many prior art aerogels require the use of tapes or other external devices in order to securely maintain the aerogel on the skin of the subject over the course of several days.

In addition, in some cases, a high degree of pore interconnectivity may allow a surprising amount of water to be absorbed into the aerogel. This may allow the aerogel to be highly adhesive, for example due to van der Waals forces from the aerogel, and thus allow the aerogel to be applied to the skin of a subject, e.g., without falling off. Moisture from the skin may be absorbed into the aerogel, and thus prevent the moisture from loosening the aerogel from the skin. In some cases, for instance, an aerogel may be able to remain adhered to the skin of a subject for at least 12 hours, at least 1 day, at least 2 days, at least 3 days, or more in certain embodiments.

Thus, in certain embodiments, an aerogel (e.g., containing one or more electrical elements) may be adhered to the skin of a subject. The adhesion may be maintained for one or more days, e.g., without using external devices such as tape to hold the aerogel in place (although in other embodiments, such devices may be used). In some cases, the electrical elements may be used to determine and/or stimulate one or more nerves within the subject. As a non-limiting example, the aerogel may be adhered to the neck, and the electrical elements used to stimulate the vagus nerve. This may be useful, for example, to treat conditions such as cachexia or wasting disease, e.g., by stimulating the parasympathetic nervous system in a pulsed manner to increase the expression of urea cycle enzymes in the liver, e.g., as discussed in Int. Pat. Apl. No. WO 2022/011222.

In addition, in certain embodiments, an aerogel may have one or more physical properties, or combinations thereof, that impart one or more desired functional properties (e.g., elasticity, adhesiveness, etc.). For example, in some embodiments, aerogels within a specific range of pore densities (e.g., between 80% and 95%), pore sizes (e.g., between 1 and 50 microns), and/or pore interconnectivities (e.g., between 65% and 90%) produce elastic aerogels (e.g., aerogels capable of recovering size and shape after deformation) Alternatively, or additionally, aerogels within a specific range of moisture permeabilities may result in adhesive aerogels (e.g., aerogels capable of adhering to a surface without the use of an known adhesive, such as double sided tape), according to certain embodiments.

As such, certain aerogels disclosed herein, according to some embodiments, may undergo reversible deformation when exposed to compressive, tensile, shear, and/or torsional stresses. Alternatively, or additionally, certain aerogels disclosed herein may be adhered to a surface of interest (e.g., a human forearm). In some cases, the aerogels adhered to a surface of interest may absorb a fluid disposed on the surface without significantly altering the adhesive strength between the aerogel and the surface. In some embodiments, however, adhesion to the surface of interest may require the use of adhesives (e.g., double-sided adhesive tape), for example, for adhesion to super hydrophobic or super hydrophilic surfaces.

In some embodiments, one or more electronic elements, such as a sensor (e.g., electrochemical sensors, impedance sensors, thermocouples, electrophysiological sensors, etc.), may be disposed on the surface of and/or be at least partially contained within an aerogel disclosed herein. The electronic elements may be disposed on any surface of the aerogel, depending on the aerogel geometry, within the bulk of the aerogel, or both. In some embodiments, the sensors are configured to detect one or more physiological parameters, individually or simultaneously (e.g., blood pressure and glucose levels or heart rate and blood pressure, etc.).

Other aspects of the present disclosure generally relate to methods of manufacturing one or more aerogels described herein. In some embodiments, the method comprises using a monomer solution to form a 3-dimensional structure. Any method known in the art to form such structures may be used herein (e.g., mold casting, 3-D printing, inkjet printing, fused filament fabrication, electrospinning, electro-spraying, microfluidic, etc.). In some embodiments, for example, the monomer solution may be flash frozen (e.g., placed in liquid nitrogen, sputtered onto a liquid nitrogen cooled metal block, etc.). Alternatively, or additionally, the monomer solution may be stored at a temperature below the freezing point of the monomer solution. In some embodiments, an aqueous phase of the monomer solution is removed (e.g., via sublimation) to yield a crosslinked polymer aerogel.

As described above, certain aerogels disclosed herein comprise a crosslinked polymer aerogel. In some cases, the crosslinked polymer aerogel possesses one or more physical parameters that impart one or more desired functionalities (e.g., adhesiveness to skin and elasticity). Examples of physical parameters of the aerogel that may impart desired functionality include average pore density, average moisture permeability, average pore interconnectivity, elastic modulus, tensile stress, tensile strength, tensile failure strain, viscoelastic modulus, air permeability, and/or combinations thereof, etc. The following is a non-limiting description of the aforementioned physical parameters and exemplary ranges useful for producing various aerogels disclosed herein.

In some embodiments, a crosslinked polymer aerogel is microporous and has a plurality of pores at the surface of the aerogel (for example, as determined via imaging analysis of micrographs obtained using SEM). As used herein, the phrase “microporous” refers to any pore (e.g., of any shape) with an average diameter of less than or equal to 1000 microns. The average pore diameter of surface pores may be between 10 micron and 50 microns. In some embodiments, a microporous crosslinked polymer aerogel has an average pore diameter greater than or equal to 10 microns, greater than or equal to 15 microns, greater than or equal to 20 microns, greater than or equal to 25 microns, greater than or equal to 30 microns, greater than or equal to 35 microns, greater than or equal to 40 microns, greater than or equal to 45 microns, and greater than or equal to 50 microns at the surface of the aerogel. In other embodiments, the microporous crosslinked aerogel has an average pore diameter less than or equal to 50 microns, less than or equal to 45 microns, less than or equal to 40 microns, less than or equal to 35 microns, less than or equal to 30 microns, less than or equal to 25 microns, less than or equal to 20 microns, less than or equal to 15 microns, and less than or equal to 10 microns at the surface of the aerogel. Combinations are also possible (e.g., greater than or equal to 10 microns and less than or equal to 50 microns). Other ranges are also possible (e.g., greater than 50 microns and less than 10 microns).

A crosslinked polymer aerogel, in some cases, may have a plurality of pores with an average pore diameter within the bulk of the aerogel, for example, as determined via imaging analysis of micrographs obtained using scanning electron microscopy, or other techniques known to those of ordinary skill in the art. In some embodiments, the average pore diameter of bulk pores is between 10 micron and 50 microns. In some embodiments, the average pore diameter is greater than or equal to 10 microns, greater than or equal to 15 microns, greater than or equal to 20 microns, greater than or equal to 25 microns, greater than or equal to 30 microns, greater than or equal to 35 microns, greater than or equal to 40 microns, greater than or equal to 45 microns, and greater than or equal to 50 microns within the bulk of the aerogel. In other embodiments, the average pore diameter is less than or equal to 50 microns, less than or equal to 45 microns, less than or equal to 40 microns, less than or equal to 35 microns, less than or equal to 30 microns, less than or equal to 25 microns, less than or equal to 20 microns, less than or equal to 15 microns, and less than or equal to 10 microns within the bulk of the aerogel. Combinations are also possible (e.g., greater than or equal to 10 microns and less than or equal to 50 microns). Other ranges are also possible (e.g., greater than 50 microns and less than 10 microns).

In certain embodiments, a crosslinked polymer aerogel may have an average pore distribution, for example, as determined by as determined via imaging analysis of micrographs obtained using scanning electron microscopy. In some cases, the average pore distribution may be between 1 micron and 200 microns, between 5 microns and 150 microns, between 10 microns and 130 microns, between 15 microns and 100 microns, between 20 microns, and 80 microns, between 25 microns and 60 microns, and between 30 microns and 40 microns. Other ranges are also possible (e.g., between 25 microns and 80 microns).

The crosslinked polymer aerogels disclosed herein, in certain embodiments, may have one or more pores that connect to one or more other pores within the bulk of the aerogel (e.g., “average pore interconnectivity”). See Example 9, Assessment of Pore Interconnectivity. In some embodiments, the crosslinked polymer aerogel has an average pore interconnectivity of between 50% and 90%. The average pore interconnectivity may be greater than or equal to 50%, greater than or equal to 55%, greater than or equal to 60%, greater than or equal to 65%, greater than or equal to 70%, greater than or equal to 75%, greater than or equal to 80%, greater than or equal to 85%, and greater than or equal to 90%. The average pore interconnectivity may be less than or equal to 90%, less than or equal to 85%, less than or equal to 80%, less than or equal to 75%, less than or equal to 70%, less than or equal to 65%, less than or equal to 60%, less than or equal to 55%, and less than or equal to 50%. Combinations are also possible (e.g., greater than or equal to 50% and less than or equal to 90%). Other ranges are also possible (e.g., less than or equal to 50%, less than or equal to 40%, less than or equal to 30%, less than or equal to 20%, less than or equal to 10%, etc. or greater than or equal to 90%, greater than or equal to 95%, greater than or equal to 99%, etc.).

In one set of embodiments, a crosslinked polymer aerogel may have an average moisture permeability, for example, as determined using the ASTM E96 standard. In some embodiments, the average moisture permeability is between 2500 g/(m² day) and 3000 g/(m² day). In some cases, the average moisture permeability may be greater than or equal to 2500 g/(m² day), greater than or equal to 2600 g/(m² day), greater than or equal to 2700 g/(m² day), greater than or equal to 2800 g/(m² day), greater than or equal to 2900 g/(m² day), and greater than or equal to 3000 g/(m² day). In other cases, the average moisture permeability may be less than or equal to 3000 g/(m² day), less than or equal to 2900 g/(m² day), less than or equal to 2800 g/(m² day), less than or equal to 2700 g/(m² day), less than or equal to 2600 g/(m² day), and less than or equal to 2500 g/(m² day). Combinations are also possible (e.g., greater than or equal to 2500 g/(m² day) and less than or equal to 3000 g/(m² day)). Other ranges are also possible (less than 2500 g/(m² day) or greater than 3000 g/(m² day)).

In some embodiments, a crosslinked polymer aerogel has an average elastic modulus of between 2 kPa and 6 kPa, e.g., as determined via the slope of the linear range of a stress-strain curve. In some cases, the average elastic modulus may be greater than or equal to 2 kPa, greater than or equal to 3 kPa, greater than or equal to 4 kPa, greater than or equal to 5 kPa, and greater than or equal to 6 kPa. In other cases, the average elastic modulus may be less than or equal to 6 kPa, less than or equal to 5 kPa, less than or equal to 4 kPa, less than or equal to 3 kPa, less than or equal to 2 kPa, and less than or equal to 1 kPa. Combinations are also possible (e.g., greater than or equal to 2 kPa and less than or equal to 6 kPa). Other ranges are also possible (e.g., less than 2 kPa or greater than 6 kPa).

In some embodiments, a crosslinked polymeric aerogel may have an average elastic modulus of between 10¹ and 10⁴ Pa, e.g., as determined via rheology (e.g., G′ value). The average elastic modulus, according to certain embodiments, may be greater than or equal to 10¹ Pa, greater than or equal to 10² Pa, greater than or equal to 10³ Pa, and greater than or equal to 10⁴ Pa. In other embodiments, the average elastic modulus may be less than or equal to 10⁴ Pa, less than or equal to 10³ Pa, less than or equal to 10² Pa, and less than or equal to 10¹ Pa. Combinations are also possible (e.g., greater than or equal to 10¹ Pa and less than or equal to 10⁴ Pa). Other ranges are also possible (e.g., less than 10¹ Pa or greater than 10⁴ Pa).

According to certain embodiments, a crosslinked polymer aerogel may have an average tensile strength of between 3 kPa and 10 kPa, for example, as determined via an abrupt drop in force from a stress-strain curve. In some cases, the average tensile strength may be greater than or equal to 3 kPa, greater than or equal to 4 kPa, greater than or equal to 5 kPa, greater than or equal to 6 kPa, greater than or equal to 7 kPa, greater than or equal to 8 kPa, greater than or equal to 9 kPa, and greater than or equal to 10 kPa. In other cases, the average tensile strength may be less than or equal to 10 kPa, less than or equal to 9 kPa, less than or equal to 8 kPa, less than or equal to 7 kPa, less than or equal to 6 kPa, less than or equal to 5 kPa, less than or equal to 4 kPa, and less than or equal to 3 kPa. Combinations are also possible (e.g., greater than or equal to 3 kPa and less than or equal to 10 kPa). Other ranges are also possible (less than 3 kPa or greater than 10 kPa).

In some cases, a crosslinked polymer aerogel may have an average tensile failure strain of between 0.4 mm/mm and 1 mm/mm. The average tensile failure strain may be greater than or equal to 0.4 mm/mm, greater than or equal to 0.5 mm/mm, greater than or equal to 0.6 mm/mm, greater than or equal to 0.7 mm/mm, greater than or equal to 0.8 mm/mm, greater than or equal to 0.9 mm/mm, and greater than or equal to 1 mm/mm. In other embodiments, the average tensile failure strain may be less than or equal to 1 mm/mm, less than or equal to 0.9 mm/mm, less than or equal to 0.8 mm/mm, less than or equal to 0.7 mm/mm, less than or equal to 0.6 mm/mm, less than or equal to 0.5 mm/mm, or less than or equal to 0.4 mm/mm. Combinations are also possible (e.g., greater than or equal to 0.4 mm/mm and less than or equal to 1 mm/mm). Other ranges are also possible (e.g., less than 0.4 mm/mm or greater than 1.0 mm/mm).

The crosslinked polymer aerogels described herein, may according to some embodiments, have an average air permeability, for example, as determined using assays known in the art and described elsewhere herein (see Example 9, Air Permeability Measurements). In some embodiments, the average air permeability is between 1×10² barrer and 1×10⁸ barrer. In one set of embodiments, the air permeability is greater than or equal to 1×10² barrer, greater than or equal to 1×10³ barrer, greater than or equal to 1×10⁴ barrer, greater than or equal to 1×10⁵ barrer, greater than or equal to 1×10⁶ barrer, greater than or equal to 1×10⁷ barrer, and greater than or equal to 1×10⁸ barrer. In another set of embodiments, the average air permeability is less than or equal to 1×10⁸ barrer, less than or equal to 1×10⁷ barrer, less than or equal to 1×10⁶ barrer, less than or equal to 1×10⁵ barrer, less than or equal to 1×10⁴ barrer, less than or equal to 1×10³ barrer, and less than or equal to 1×10² barrer. Combinations are also possible (e.g., greater than or equal to 1×10² barrer and less than or equal to 1×10⁸ barrer). Other ranges are also possible (e.g., less than 1×10² barrer or greater than 1×10⁸ barrer).

In some cases, any one of the crosslinked polymer aerogels described herein, may have an average compressive fracture stress between 50 kPa and 200 kPa. In some embodiments, the compressive fracture stress is the maximum stress at which the stress vs strain curve precipitously drops to zero. In some cases, the average compressive fracture stress of the aerogel may be greater than or equal to 50 kPa, greater than or equal to 75 kPa, greater than or equal to 100 kPa, greater than or equal to 125 kPa, greater than or equal to 150 kPa, greater than or equal to 175 kPa, and greater than or equal to 200 kPa. In some embodiments, the average compressive fracture stress of the aerogel is less than or equal to 200 kPa, less than or equal to 175 kPa, less than or equal to 150 kPa, less than or equal to 125 kPa, less than or equal to 100 kPa, less than or equal to 75 kPa, and less than or equal to 50 kPa. Combinations are also possible (e.g., greater than or equal to 50 kPa and less than or equal to 200 kPa). Other ranges are also possible (e.g., less than 50 kPa or greater than 200 kPa).

Any one of the crosslinked polymer aerogels disclosed herein may have an average compressive modulus of between 5 kPa and 100 kPa. In some cases, the average compressive modulus of the aerogel may be greater than or equal to 5 kPa, greater than or equal to 10 kPa, greater than or equal to 25 kPa, greater than or equal to 50 kPa, greater than or equal to 75 kPa, and greater than or equal to 100 kPa. In some embodiments, the average compressive fracture stress of the aerogel is less than or equal to 100 kPa, less than or equal to 75 kPa, less than or equal to 50 kPa, less than or equal to 25 kPa, less than or equal to 10 kPa, and less than or equal to 5 kPa. Combinations are also possible (e.g., greater than or equal to 5 kPa and less than or equal to 100 kPa). Other ranges are also possible (e.g., less than 5 kPa or greater than 100 kPa).

In certain embodiments, any one of the crosslinked polymer aerogel disclosed herein may have an average compressive failure strain of between 40% and 95% . . . . In some embodiments, the compressive fracture strain is the maximum strain at which the stress vs strain curve precipitously drops to zero. In some embodiments, the average compressive failure strain is greater than or equal to 40%, greater than or equal to 50%, greater than or equal to 60%, greater than or equal to 70%, greater than or equal to 80%, greater than or equal to 90%, and greater than or equal to 95%. According to other embodiments, the average compressive failure strain is less than or equal to 95%, less than or equal to 90%, less than or equal to 80%, less than or equal to 70%, less than or equal to 60%, less than or equal to 50%, and less than or equal to 40%. Combinations are also possible (e.g., greater than or equal to 40% and less than or equal to 95%) . . . . Other ranges are also possible (e.g., less than 40% or greater than 95%).

Any one of the aerogels disclosed herein, may be repeatedly stretched. Thus, in some embodiments, the article may hysterese (e.g., undergo hysteresis), for example, as determined by cyclic tensile testing using an Instron instrument. In some cases, the crosslinked polymer aerogel undergoes an average of between 1% and 20% hysteresis following at least 1 cycle of a cyclic tensile test. In some cases, the aerogel may undergo an average of greater than or equal to 1% hysteresis, greater than or equal to 5% hysteresis, greater than or equal to 10% hysteresis, greater than or equal to 15% hysteresis, and greater than or equal to 20% hysteresis following at least 1 cycle of a cyclic tensile test. In certain cases, the aerogel may undergo an average of less than or equal to 20% hysteresis, less than or equal to 15% hysteresis, less than or equal to 10% hysteresis, less than or equal to 5% hysteresis, and less than or equal to 1% hysteresis following at least 1 cycle of a cyclic tensile test. Combinations are also possible (e.g., greater than or equal to 1% hysteresis and less than or equal to 20% hysteresis). Other ranges are also possible (e.g., less than 1% hysteresis or greater than 20% hysteresis).

In some cases, the crosslinked polymer aerogel may undergo an average of between 1% and 20% hysteresis following at least 10 cycles of a cyclic tensile test. In some cases, the aerogel may undergo an average of greater than or equal to 1% hysteresis, greater than or equal to 5% hysteresis, greater than or equal to 10% hysteresis, greater than or equal to 15% hysteresis, and greater than or equal to 20% hysteresis following at least 10 cycles of a cyclic tensile test. In certain cases, the aerogel may undergo an average of less than or equal to 20% hysteresis, less than or equal to 15% hysteresis, less than or equal to 10% hysteresis, less than or equal to 5% hysteresis, and less than or equal to 1% hysteresis following at least 10 cycles of a cyclic tensile test. Combinations are also possible (e.g., greater than or equal to 1% hysteresis and less than or equal to 20% hysteresis). Other ranges are also possible (e.g., less than 1% hysteresis or greater than 20% hysteresis).

In some cases, the crosslinked polymer aerogel may undergo an average of between 1% and 20% hysteresis following at least 50 cycles of a cyclic tensile test. In some cases, the aerogel may undergo an average of greater than or equal to 1% hysteresis, greater than or equal to 5% hysteresis, greater than or equal to 10% hysteresis, greater than or equal to 15% hysteresis, and greater than or equal to 20% hysteresis following at least 50 cycles of a cyclic tensile test. In certain cases, the aerogel may undergo an average of less than or equal to 20% hysteresis, less than or equal to 15% hysteresis, less than or equal to 10% hysteresis, less than or equal to 5% hysteresis, and less than or equal to 1% hysteresis following at least 50 cycles of a cyclic tensile test. Combinations are also possible (e.g., greater than or equal to 1% hysteresis and less than or equal to 20% hysteresis). Other ranges are also possible (e.g., less than 1% hysteresis or greater than 20% hysteresis).

In some cases, the crosslinked polymer aerogel may undergo an average of between 1% and 20% hysteresis following at least 100 cycles of a cyclic tensile test. In some cases, the aerogel may undergo an average of greater than or equal to 1% hysteresis, greater than or equal to 5% hysteresis, greater than or equal to 10% hysteresis, greater than or equal to 15% hysteresis, and greater than or equal to 20% hysteresis following at least 100 cycles of a cyclic tensile test. In certain cases, the aerogel may undergo an average of less than or equal to 20% hysteresis, less than or equal to 15% hysteresis, less than or equal to 10% hysteresis, less than or equal to 5% hysteresis, and less than or equal to 1% hysteresis following at least 100 cycles of a cyclic tensile test. Combinations are also possible (e.g., greater than or equal to 1% hysteresis and less than or equal to 20% hysteresis). Other ranges are also possible (e.g., less than 1% hysteresis or greater than 20% hysteresis).

Any one of the aerogels disclosed herein, in some instances, may absorb a fluid (e.g., a body fluid) into the crosslinked polymer framework of the aerogel as determined by mass analysis. In some cases, the aerogel may absorb an average volume of fluid equal to between 500% w/w and 1000% w/w (relative to the dry weight of the crosslinked polymer aerogel). In some instances, the average absorbed volume of fluid may be greater than or equal to 500% w/w, greater than or equal to 600% w/w, greater than or equal to 700% w/w, greater than or equal to 800% w/w, greater than or equal to 900% w/w, and greater than or equal to 1000% w/w, relative to the dry weight of the crosslinked polymer aerogel. In other cases, the average absorbed volume of fluid may be less than or equal to 1000% w/w, less than or equal to 900% w/w, less than or equal to 800% w/w, less than or equal to 700% w/w, less than or equal to 600% w/w, and less than or equal to 500% w/w. Combinations are also possible (e.g., greater than or equal to 500% w/w and less than or equal to 1000% w/w).

In some embodiments, any one of the aerogels disclosed herein, may swell upon absorbing a fluid, for example, as determined via the fractional increase in weight of the aerogel due to water absorption. The degree to which the aerogel may swell may vary between 1% and 20% (the percentage is a volume percent relative to the initial volume of the aerogel). In some instances, the aerogel may swell by greater than or equal to 1%, greater than or equal to 2%, greater than or equal to 3%, greater than or equal to 4%, greater than or equal to 5%, greater than or equal to 6%, greater than or equal to 7%, greater than or equal to 8%, greater than or equal to 9%, greater than or equal to 10%, greater than or equal to 15%, and greater than or equal to 20% when absorbing between 500% w/w and 1000% w/w of a fluid (e.g., saline). In other instances, the aerogel may swell by less than or equal to 20%, less than or equal to 15%, less than or equal to 10%, less than or equal to 9%, less than or equal to 8%, less than or equal to 7%, less than or equal to 6%, less than or equal to 5%, less than or equal to 4%, less than or equal to 3%, less than or equal to 2%, and less than or equal to 1%, when absorbing between 500% w/w and 1000% w/w of a fluid (e.g., saline). Combinations are also possible (e.g., greater than or equal to 1% and less than or equal to 20%). Other ranges are also possible (e.g., less than 1% or greater than 20%).

Aerogels of the present disclosure may, in some embodiments, comprise a polymer. Any suitable polymer (e.g., capable of being crosslinked) known to one of skill in the art may be used to produce any one of the aerogels disclosed herein. In some cases, the polymer is a biopolymer, for example, a polypeptide (e.g., collagen, silk fibroin, gelatin, chitosan, hyaluronic acid, etc.), a nucleic acid (DNA, RNA, etc.), or a carbohydrate (e.g., dextran, starch, cellulose, and alginate, etc.). Other polymers are also contemplated. For instance, in some cases, aerogels may be fabricated using biodegradable polymers such as polyesters, polyanhydrides, polyurethanes, poly(ester amides), polyethylene glycol, silica, and poly(glycerol sebacate). Combinations are also possible. For example, a polymer may comprise more than one type of polymer (e.g., a copolymer) and may be arranged in more than one configuration (e.g., random copolymer, block copolymer, triblock copolymer, etc.). Additionally, physical blends of polymers are also possible.

Aerogels of the present disclosure may have an electronic element disposed on one or more surfaces of the aerogel. In some embodiments, the aerogel may comprise at least one electronic element, at least two electronic elements, at least three electronic elements, at least four electronic elements, at least five electronic elements, at least six electronic elements, at least seven electronic elements, at least eight electronic elements, at least nine electronic elements, and at least ten electronic elements on a surface of the aerogel.

The electronic element may be positioned on any surface of any one of the aerogels disclosed herein. For example, aerogels with a square planar structure (e.g., two flat opposing surfaces) may have at least one electronic element on a first surface, a second surface opposing the first surface, or on both surfaces. In addition, the electronic element may be positioned in an aerogel in other embodiments.

Other aspects of the present disclosure relate to methods for producing an article (e.g., an aerogel) disclosed herein. In some embodiments, the method comprises preparing a monomer solution capable of being crosslinked using a polymerization reaction. The monomer solution may comprise one or more monomers bearing one or more reactive groups capable of undergoing a polymerization reaction. In some cases, the monomer is a polymer (e.g., gelatin) bearing one or more reactive groups capable of undergoing a polymerization reaction.

Without wishing to be bound by theory, the polymerization reaction may proceed by any mechanism known in the art, for example, chain-growth polymerization (e.g., radical polymerization, coordinative polymerization, ionic polymerization, living polymerization, ring-opening polymerization, and reversible-deactivation polymerization), condensation/step-growth polymerization (e.g., polyesters, polyamides, polyurethanes, polyurea, polysiloxane, polycarbonates, polysulfides, polyether, etc.), or photopolymerization.

In some embodiments, a monomer solution comprises one or more monomers dissolved in an aqueous medium (e.g., water, saline, etc.) at a final concentration of between 1% and 10% w/w (e.g., relative to the total weight of the monomer solution). The final monomer concentration in the monomer solution may be greater than or equal to 1%, greater than or equal to 2%, greater than or equal to 3%, greater than or equal to 4%, greater than or equal to 5%, greater than or equal to 6%, greater than or equal to 7%, greater than or equal to 8%, greater than or equal to 1%, greater than or equal to 9%, and greater than or equal to 10% of the total weight of the monomer solution (w/w). The final monomer concentration in the monomer solution may be less than or equal to 10%, less than or equal to 9%, less than or equal to 8%, less than or equal to 7%, less than or equal to 6%, less than or equal to 5%, less than or equal to 4%, less than or equal to 3%, less than or equal to 2%, and less than or equal to 1% of the total weight of the monomer solution (w/w). In one set of embodiments, the final monomer concentration in the monomer solution is greater than or equal to 3% w/wand less than or equal to 10% w/w. Combinations are also possible (e.g., greater than or equal to 1% w/w and less than or equal to 10% w/w). Other ranges are also possible (e.g., less than 1% w/w and greater than 10% w/w).

Exemplary embodiments of reactive groups for use in chain-growth polymerization reactions include, but are not limited to, alkenes and/or functionally substituted alkenes (e.g., allyl groups, vinyl groups, etc.). In some embodiments, the preferred reactive group is methacrylic acid (e.g., derived from methacrylic anhydride); in other embodiments, the preferred reactive group is acrylic acid (e.g., derived from acrylic anhydride).

Exemplary embodiments of reactive groups for use in step-growth polymerization reactions include, but are not limited to, any group capable of reacting with a second reactive group to form a covalent bond, such as those between electrophiles (e.g., maleimides, N-hydroxysuccinimide (NHS) activated esters, carbodiimides, hydrazide, tetrafluorophenyl (TFP) esters, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctynes, and aldehydes) and neutrophiles (e.g., free amines (—NH2), free sulfhydryl groups (—SH), free hydroxide groups (—OH), carboxylates, hydrazides, and alkoxyamines), etc.

In some embodiments, the reactive groups comprise chemical functionalities used in “click” chemistry. “Click” chemistry comprises the reaction between a functional group with a 1,3-dipole, such as an azide, a nitrile oxide, a nitrone, an isocyanide, and the like, with an alkene or an alkyne dipolarophiles. Exemplary dipolarophiles include any strained cycloalkenes and cycloalkynes, including, but not limited to, cyclooctynes, dibenzocyclooctynes, monofluorinated cyclcooctynes, difluorinated cyclooctynes, and biarylazacyclooctynone.

In some embodiments, a monomer comprises a polyethylene glycol (PEG) linker separating the reactive group from the monomer backbone. Non-limiting examples functional PEGs linkers that may be purchased by commercial vendors, or synthetized by those skilled in the art include OH-PEG-SC, OH-PEG-SCM, OH-PEG-SG, OH-PEG-SS, SH-PEG-OH, SH-PEG-NH2, SH-PEG-COOH, SH-PEG-HZ, SH-PEG-EPO, SH-PEG-SC, SH-PEG-SCM, SH-PEG-SG, SH-PEG-SS, SH-PEG-GAS, SH-PEG-LA, SH-PEG-Biotin, SH, PEG-AA, CHO-PEG-OH, CHO-PEG-SH, CHO-PEG-NH2, CHO-PEG-N3, CHO-PEG-MA, CHO-PEG-COOH, CHO-PEG-PEO, CHO-PEG-Mal, CHO-PEG-SC, CHO-PEG-SCM, CHO-PEG-SS, NH2-PEG-EPO, NH2-PEG-DBCO, N3-PEG-OH, N3-PEG-SH, N3-PEG-NH2, N3-PEG-alkene, N3-PEG-EPO, N3-PEG-Mal, N3-PEG-NHS, N3-PEG-SCM, N3-PEG-SG, N3-PEG-SS, N3-PEG-Biotin, Alkyene-PEG-OH, Alkyene-PEG-SH, Alkyene-PEG-CHO, Alkyene-PEG-NH2, Alkyene-PEG-N3, Alkyene-PEG-MA, Alkyene-PEG-COOH, Alkyne-PEG-EPO, Alkyene-PEG-Mal, Alkyene-PEG-SC, Alkyene-PEG-biotin, Alkene-PEG-OH, AC-PEG-OH, AC-PEG-SH, AC-PEG-CHO, AC-PEG-NH2, AC-PEG-N3, AC-PEG-Alkyene, AC-PEG-MA, AC-PEG-EPO, AC-PEG-MAL, AC-PEG-SC, AC-PEG-SCM, AC-PEG-SG, AC-PEG-SS, AC-PEG-SVA, AC-PEG-SS-PEG-AC, ACA-PEG-OH, ACA-PEG-SH, ACA-PEG-SH, ACA-PEG-NH2, ACA-PEG-N3, ACA-PEG-SA, ACA-PEG-COOH, ACA-PEG-NH-MAL, ACA-PEG-SC, ACA-PEG-SCM, ACA-PEG-SG, ACA-PEG-SS, MA-PEG-OH, MA-PEG-SH, MA-PEG-NH2, MA-PEG-N3, MA-PEG-EPO, MA-PEG-MAL, MA-PEG-SC, COOH-PEG-N3, COOH-PEG-MA, COOH-PEG-HZ, COOH-PEG-PEO, COOH-PEG-SS, MAL-PEG-OH, MAL-PEG-NH2, MAL-PEG-COOH, MAL-PEG-HZ, MAL-PEG-SC, MAL-PEG-SCM, MAL-NH-PEG-SPA, MAL-PEG-SG, MAL-PEG-SS, MAL-PEG-SVA, MAL-PEG-PFP, SC-PEG-Alkene, NHS-PEG-COOH, SCM-PEG-COOH, SG-PEG-COOH, OPSS-PEG-OH, OPSS-PEG-NH2, OPSS-PEG-COOH, OPSS-PEG-MAL, OPSS-PEG-NHS, OPSS-PEG-SCM, OPSS-PEG-biotin, LA-PEG-OH, LA-PEG-NH2, LA-PEG-N3, LA-PEG-COOH, LA-PEG-MAL, LA-PEG-SC, LA-PEG-SCM, LA-PEG-SG, LA-PEG-SS, LA-PEG-biotin, biotin-PEG-OH, biotin-PEG-CHO, biotin-PEG-NH2, biotin-PEG-AC, biotin-PEG-COOH, biotin-PEG-HZ, biotin-PEG-MAL, biotin-PEG-NHS, biotin-PEG-SCM, biotin-PEG-SC, biotin-PEG-SS, biotin-PEG-SAS, biotin-PEG-SVA, biotin-PEG-PFP, bis-biotin-PEG-TFP, DBCO-PEG-OH, DBCO-PEG-SH, DBCO-PEG-COOH, DBCO-PEG-MAL, DBCO-PEG-NHS, and DBCO-PEG-biotin. In some embodiments, the functional linkers comprising polyethylene glycol comprise X-PEG-N3, X-PEG-DBCO, X-PEG-biotin, X-PEG-avidin, X-PEG streptavidin, X-PEG-COOH, or X-PEG-SH, wherein X is an activated ester, an isocyanate, or thioisocyanate.

In some embodiments, multiple functional groups on a functional linker are separated by a polymer other than polyethylene glycol. In some embodiments, the polymer is a linear polymer, a branched polymer and/or a polymer brush.

In some instances, the monomer solution may be heated, for example, to aid in dissolution of the monomer into the solution phase. The monomer solution may be heated to any suitable concentration to aid in the dissolution of the monomer, in so long as doing so does not degrade the chemical structure of the monomer. In some embodiments, the monomer solution may be heated to greater than or equal to 37° C., greater than or equal to 40° C., greater than or equal to 45° C., greater than or equal to 50° C., greater than or equal to 55° C., greater than or equal to 60° C., greater than or equal to 65° C., greater than or equal to 70° C., greater than or equal to 75° C., and greater than or equal to 80° C. In other embodiments, the monomer solution may be heated to less than or equal to 80° C., less than or equal to 75° C., less than or equal to 70° C., less than or equal to 65° C., less than or equal to 60° C., less than or equal to 55° C., less than or equal to 50° C., less than or equal to 45° C., less than or equal to 40° C., and less than or equal to 37° C. In one set of embodiments, the monomer solution is heated to greater than or equal to 60° C. and less than or equal to 80° C. Other combinations are also possible (e.g., greater than or equal to 37° C. and less than or equal to 80° C.). Other ranges are also possible (e.g., less than 37° C. or greater than 80° C.).

The monomer solution, in some cases, may comprise one or more initiators, for example, to start the polymerization reaction (e.g., such as may be needed for chain-growth polymerizations and photopolymerizations). Non-limiting examples that may be purchased by commercial vendors (e.g., TCI) or synthesized by known methods include ammonium persulfate (APS), 2,2′-azobis [2-(2-imidazolin-2-yl)-propane] dihydrochloride, TBHP, cumene hydroperoxide, di-tert-butyl peroxide, dicumyl peroxide, BPO, dicyandiamide, cyclohexyl tosylate, (4-hydroxyphenyl)-dimethylsulfonium hexafluorophosphate, diphenyl(methyl) sulfonium tetrafluoroborate, benzyl (4-hydroxyphenyl)-methylsulfonium hexafluoroantimonate, (4-hydroxyphenyl)methyl-(2-methylbenzyl) sulfonium hexafluoroantimonate, triphenylsulphonium nonaflate.

In some embodiments, the initiator is a photopolymerization initiator, such as a photo-radical initiator, a photo-cationic initiator, or a photo-anionic initiator. Non-limiting examples that may be purchased by commercial vendors (e.g., TCI, 2-Hydroxy-4′-(2-hydroxyethoxy)-2-methylpropiophenone, and lithium phenyl-2,4,6-trimethylbenzoylphosphinate) or synthesized by known methods include (+)-Camphorquinone, Acetophenone, 4′-Hydroxyacetophenone, 3-Hydroxybenzophenone, 4,4′-Dihydroxybenzophenone, 4′-Hydroxyacetophenone, 3′-Hydroxyacetophenone, Benzophenone, 3-Methylbenzophenone, 2-Methylbenzophenone, 3,4-Dimethylbenzophenone, 3-Hydroxybenzophenone, 4-Hydroxybenzophenone, 4,4′-Dihydroxybenzophenone, 4-Benzoylbenzoic Acid, 2-Benzoylbenzoic Acid, Methyl 2-Benzoylbenzoate, 4,4′-Carbonyldiphthalic Anhydride, 4-(Dimethylamino)-benzophenone, 4,4′-Bis(dimethylamino)-benzophenone, 4,4′-Bis(diethylamino)-benzophenone, 4,4′-Dichlorobenzophenone, 4-Phenylbenzophenone, Diphenyliodonium Hexafluoroarsenate, [4-[(2-Hydroxytetradecyl)-oxy]phenyl]phenyliodonium Hexafluoroantimonate, 1,2-Bis(4-methoxyphenyl)-2-oxoethyl Cyclohexylcarbamate, and Nifedipine. Other initiators are also possible. Other additives are also possible. For example, in some embodiments a catalyst may be added to the monomer solution. In some cases, the catalyst is tetramethylethylenediamine (TEMED).

In some embodiments, a monomer solution comprises one or more initiators at a final concentration of between 0.1% and 5% w/v (e.g., relative to the total volume of the monomer solution). The final initiator concentration in the monomer solution may be greater than or equal to 0.1%, greater than or equal to 0.2%, greater than or equal to 0.3%, greater than or equal to 0.4%, greater than or equal to 0.5%, greater than or equal to 0.6%, greater than or equal to 0.7%, greater than or equal to 0.8%, greater than or equal to 0.9%, greater than or equal to 1%, and greater than or equal to 5% of the total volume of the monomer solution (w/v). The final initiator concentration in the monomer solution may be less than or equal to 5%, less than or equal to 1%, less than or equal to 0.9%, less than or equal to 0.8%, less than or equal to 0.7%, less than or equal to 0.6%, less than or equal to 0.4%, less than or equal to 0.3%, less than or equal to 0.2%, and less than or equal to 0.1% of the total volume of the monomer solution (w/v). In a preferred set of embodiments, the final initiator concentration in the monomer solution is greater than or equal to 0.5% w/v and less than or equal to 1% w/v. Combinations are also possible (e.g., greater than or equal to 0.1% w/v and less than or equal to 5% w/v). Other ranges are also possible (e.g., less than 0.1% w/v or greater than 5% w/v).

In some instances, the method comprises forming a 3-dimensional structure using the monomer solution. Any technique known in the art may be used to form the 3-dimensional structure, such as cast molding, 3D-printing, inkjet printing, microfluidic, electrospinning, electro-spraying, etc. Other methods may also be used in other embodiments. In one set of embodiments, the method comprises forming the 3-dimensional structure using cast molding, e.g., by pouring the monomer solution into a mold, such as, a PDMS mold, metal mold or Teflon mold. The mold may be of any geometry known to one of skill in the art. Exemplary geometries include, but are not limited to, a polyhedron (e.g., regular tetrahedron, toroidal, rhombic triacontahedron, great cubicuboctahedron, icosidodecahedron, small stellated dodecahedron, skeletal polyhedron), a cone, sphere, or cylinder. Other geometries are also possible in other embodiments (e.g., complex shapes with irregular geometries).

In some embodiments, the mold is pre-cooled prior to adding the monomer solution. In some cases, the mold is pre-cooled to a temperature between −20° C. (e.g., using a standard freezer) and −196° C. (e.g., using liquid nitrogen). For example, the mold may be pre-cooled to a temperature greater than or equal to −150° C., greater than or equal to −125° C., greater than or equal to −100° C., greater than or equal to −80° C., greater than or equal to −60° C., greater than or equal to −50° C., greater than or equal to −30° C., and greater than or equal to −20° C. In other instances, the mold may be pre-cooled to a temperature less than or equal to −20° C., less than or equal to −30° C., less than or equal to −50° C., less than or equal to −60° C., less than or equal to −80° C., less than or equal to −100° C., less than or equal to −125° C., less than or equal to −150° C., and less than or equal to −196° C.

In certain embodiments, the 3-dimensional structure is flash frozen, for example, using liquid nitrogen. Other methods of flash freezing are possible in other embodiments. In some cases, a mold containing the monomer solution is flash frozen to form the 3-dimensional structure. Alternatively, or additionally, in some embodiments, the 3-dimensional structure may be stored at a temperature between −20° C. and −80° C. for between 1 hour and 24 hours. In some cases, the mold is stored at a temperature greater than or equal to −80° C., greater than or equal to −70° C., greater than or equal to −60° C., greater than or equal to −50° C., greater than or equal to −40° C., greater than or equal to −30° C., and greater than or equal to −20° C. after flash freezing in liquid nitrogen. In other embodiments, the mold is stored at a temperature of less than or equal to −20° C., less than or equal to −30° C., less than or equal to −40° C., less than or equal to −50° C., less than or equal to −60° C., less than or equal to −70° C., and less than or equal to −80° C. after flash freezing in liquid nitrogen. Combinations are also possible (e.g., greater than or equal to −80° C. and less than or equal to −20° C.). Other ranges are also possible (e.g., less than −80° C. or greater than −20° C.).

The mold containing the monomer solution may be stored at a temperature of between −20° C. and −80° C. for between 1 hour and 24 hours, according to some embodiments. Thus, in some cases, the mold is stored at a temperature of between −20° C. and −80° C. for greater than or equal to 1 hour, greater than or equal to 5 hours, greater than or equal to 10 hours, greater than or equal to 15 hour, greater than or equal to 20 hours, greater than or equal to 24 hours after flash freezing in liquid nitrogen. The mold, in other embodiments, be stored at a temperature of between −20° C. and −80° C. for less than or equal to 24 hours, less than or equal to 20 hours, less than or equal to 15 hours, less than or equal to 10 hours, less than or equal to 5 hours, and less than or equal to 1 hour. Other combinations are also possible (e.g., greater than or equal to 1 hour and less than or equal to 24 hours). Other ranges are possible (e.g., less than 1 hour or greater than 24 hours).

Without wishing to be bound by theory, it is believed that flash freezing the monomer solution in liquid nitrogen performs functions such as: (1) it forms a dense interconnected network of nano- and micron-sized ice crystals and/or (2) it slows the reaction kinetics of the polymerization reaction such that the monomer solution polymerizes around the interconnected network of ice crystals. The combination of these actions produces interpenetrating networks of ice crystals and crosslinked monomers (herein referred to as a “cryogel”) within the frozen aqueous medium.

In some embodiments, the method comprises removing the frozen aqueous medium from the cryogel via sublimation (e.g., freeze-drying, or supercritical drying). Subliming the frozen aqueous medium removes the network of ice crystals as well as the bulk of the weight of the cryogel (e.g., between 90% and 99% w/w) to yield a crosslinked polymer network with high porosity and pore interconnectivity (herein referred to as an aerogel).

The following are each incorporated herein by reference in their entireties: U.S. Provisional Patent Application Ser. No. 63/433,630, filed Dec. 19, 2022, entitled “Aerogel Compositions and Methods;” U.S. Provisional Patent Application Ser. No. 63/434,210, filed Dec. 21, 2022, entitled “Aerogel Compositions and Methods;” U.S. Provisional Patent Application Ser. No.: 63/433,642, filed Dec. 19, 2022, entitled “Flexible Electronics Systems for the Skin and Other Applications;” and U.S. Provisional Patent Application Ser. No. 63/434,214, filed Dec. 21, 2022, entitled “Flexible Electronics Systems for the Skin and Other Applications.”

The following examples are intended to illustrate certain embodiments of the present disclosure, but do not exemplify the full scope of the disclosure.

### EXAMPLES

### Example 1. A Breathable, Passive-Cooling, Non-Inflammatory, and Biodegradable Aerogel Electronics for Wearable Physical-Electrophysical-Chemical Analysis

Real-time monitoring of human health can be significantly improved by designing novel electronic skin (E-skin) platforms that mimic the characteristics and sensitivity of human skin. A high-quality E-skin platform that can simultaneously monitor multiple physiological signals and metabolic biomarkers without introducing skin irritation is an unmet medical need. Conventional E-skins are either bulky or made from elastomeric films that do not include key synergistic features of natural skin, such as porosity, breathability, and thermal management capabilities in a single patch. Herein, a biocompatible and biodegradable E-skin patch based on gelatin methacryloyl (GelMA) aerogel for non-invasive and continuous monitoring of multiple physiological parameters was engineered and demonstrated. Taking advantage of cryogenic temperature treatment and slow polymerization, a flexible GelMA aerogel (FGA) patches with highly interconnected porous structure that can absorb and maintain body fluids, integrate into the skin, and exhibit sensory performance under cyclic loading was fabricated. This highly interconnected porous structure of FGA gives the E-skin patch good flexibility, high passive-cooling capabilities, and ultra-lightweight properties that make it comfortable to wear for long periods of time. It also provides numerous permeable capillary channels for thermal-moisture transfer, ensuring excellent breathability. Therefore, the engineered FGA-based E-skin can simultaneously monitor body temperature, hydration, and biopotentials via electrophysiological sensors and detect multiple biomarkers such as glucose, lactate, and alcohol via electrochemical sensors. This work offers a previously unexplored materials strategy for next-generation E-skin platform with superior practicability.

Human skin is the largest organ of the body, providing an essential somatosensory ecosystem for humans to perceive, and communicate with the physical world. Developing electronic skins (E-skins) that mimic features and functionalities of natural skin hold promise in widespread applications, including healthcare monitoring and management, intelligent human-machine interfaces, and precision medicine. The E-skin typically consists of a sensor layer and a substrate. The sensor transduces biomarker information into electrical signals. Whereas the substrate offers flexibility, stretchability, and tolerance to conditions such as high temperature, humidity, and mechanical deformation. In particular, ideal E-skins should integrate multiplexed sensing capabilities on a single wearable device to provide greater insight into the users' physiological status without hampering their routine or introducing discomfort.

To date, various E-skins have been reported for unobtrusive monitoring of single or multiple physical/physiological parameters, including biopotentials (i.e., electrocardiogram, ECG; electroencephalogram, EEG; or electromyography, EMG), body temperature, human motion, skin hydration as well as a library of metabolic markers (such as glucose, insulin, lactate, and cortisol). However, most E-skins are limited to conventional elastomeric substrates (e.g., polydimethylsiloxane, PDMS; polyimide; polyethylene terephthalate; and Ecoflex) which suffer from one or more drawbacks, including mechanical mismatch, poor biocompatibility, and low permeability. Such limitations prevent sweat evaporation off the skin and limit the emission of volatile organic components (VOCs), which lead to skin irritation and inflammation. Also, typical elastomers are neither disposable nor recyclable, leading to more plastic waste and increased environmental burden. The next generation of high-quality E-skin devices with enhanced comfort and practicality should therefore be breathable, biodegradable, biocompatible, and withstand mechanical deformation especially for long-term on-body use.

To address these challenges comprehensively, materials design for novel E-skin substrates are required. One of the leading candidates is gelatin methacryloyl (GelMA) hydrogel, which is a cost-effective, naturally derived polymer from skin collagen. GelMA hydrogels have been widely used in tissue engineering, bioprinting, and implantations, because of their similarities to extracellular matrix (ECM) microenvironments and biocompatibility, biodegradability, bioadhesion, and tissue-like mechanical properties. However, GelMA can offer additional features compared to its hydrogel forms, such as high porosity, anti-swelling and ultra-lightweight properties, if used in an aerogel form. Despite some applications of GelMA hydrogel in wearable electronic devices, GelMA aerogel has not previously been explored in E-skin applications due to its natural brittleness when in a dry state. Therefore, in order to use GelMA for wearable E-Skin applications, it is essential to develop a strategy to prepare flexible GelMA aerogel (FGA) without sacrificing the abovementioned advantageous features of GelMA.

Here, a novel fabrication strategy was explored by cryofreezing of GelMA pre-polymer in liquid nitrogen followed by slow chemical polymerization at low temperatures (−80° C.) to provide flexibility to the GelMA aerogel. Furthermore, numerous three-dimensional (3D) capillary porous channels in FGA substrate offer efficient thermal-moisture transfer that ensures breathable capabilities for dynamic and comprehensive health-monitoring. Herein, it is demonstrated that soft sensor arrays can be integrated into the wearable FGA via scalable screen-printing techniques. Electrophysiological sensors were screen-printed on FGA substrate to monitor skin temperature, skin impedance/hydration levels, and ECG patterns continuously, while electrochemical sensors were integrated to characterize the levels of metabolic biomarkers (i.e., lactate, glucose, and alcohol). The engineered FGA-based E-skin can be in direct contact with the skin and simultaneously sense while stimulating sweat excretion and extracting interstitial fluid (ISF) through reverse iontophoresis. This FGA-based E-skin can detect dynamic physical, physiological, and metabolic responses to daily activities, including food/alcohol intake and exercise, as well as facilitate the early prediction of abnormal vital-sign changes. FGA with passive-cooling and E-skin functionality for multiplexed chemical-electrophysiological-physical analyses has not been reported previously.

### Example 2. Electronic Design, Device Fabrication, and In Vitro Validation

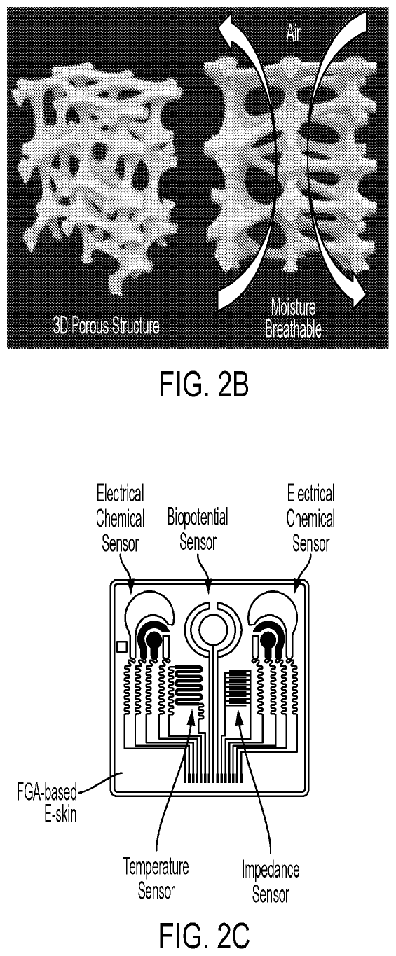

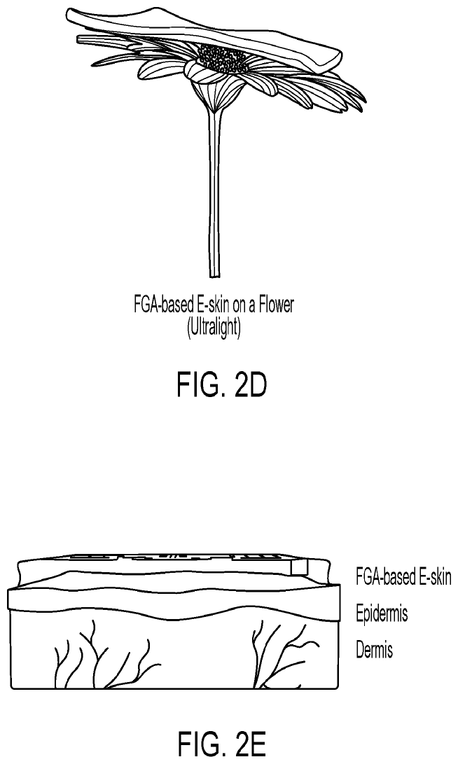

FIG. 2A schematically shows an overview of the FGA-based E-skin patch worn on the skin. A 3D porous structure of FGA-based E-skin enables its outstanding breathability performance. Air and moisture can easily pass through the interconnected capillary microchannels to balance the thermal-moisture equilibrium of the microenvironment between human skin and the outer environment (FIG. 2B). The sensor arrays and serpentine interconnections were screen-printed onto the flexible and conformal aerogel substrate to construct an FGA-based E-skin. (FIGS. 10-11). FIG. 2C briefly introduces the multimodal sensing functionality of the FGA-based E-skin, including the skin impedance/hydration sensor, temperature sensor, biopotential, and electrochemical sensors. A digital photograph of the prepared FGA-based E-skin resting on a flower is exhibited in FIG. 2D, indicating its ultra-lightweight (approximately 200 mg) and extremely low densities (approximately 0.03 g/cm³). FIG. 2E displays the conformal capability of FGA-based E-skin on the skin.

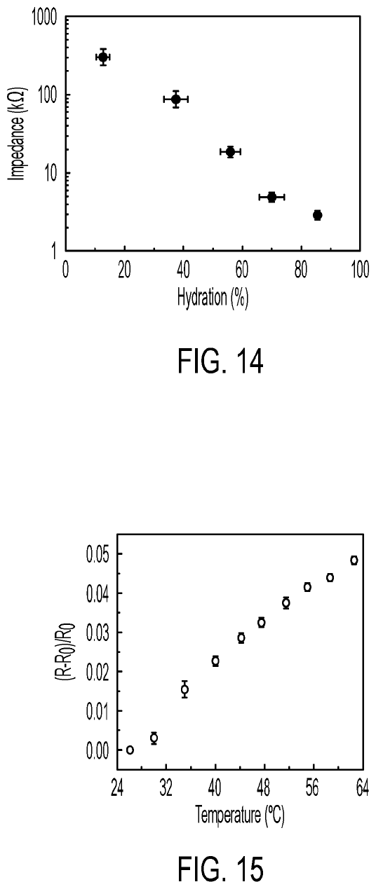

An impedance sensor for measuring the hydration level of the skin was designed, as follows. Two interdigitated electrodes were placed on the epidermis surface. Interdigitated patterns were chosen to maximize the interactions between the electrodes in a relatively small area. Fringing fields generated by the electrodes penetrate the upper skin layer, as shown conceptually in FIG. 10 and described in EXAMPLE 8. The conductive ink was screen-printed in an interdigitated pattern with a finger spacing of 200 μm. In addition, the flexibility of the GelMA aerogel substrate enables conformal and soft contact with the skin surface. The impedance sensing capabilities of the FGA-based E-skin were characterized in vitro using a gelatin-based skin phantom. First, impedance frequency sweeps were carried out across various hydration states as determined by a commercial hydration meter (FIG. 13). The measured impedance decreases as the skin phantom dries out because of its lower water content. FIG. 14 demonstrates the average impedance at 100 kHz for each case plotted against the standard values of the commercial hydration meter.

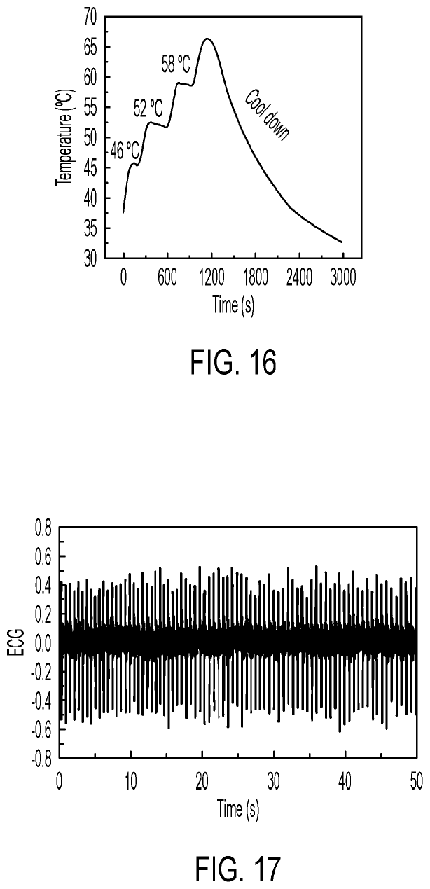

To measure skin temperature, we used the thermoresistive effect, which leads to changes in the resistance of conductive materials as temperature varies due to thermally enhanced charge transport (resistance decreases) or thermally introduced charge carrier scattering (resistance increases). Silver/silver chloride (Ag/AgCl) conductive ink was used to print the temperature sensor because Ag has a high thermal coefficient of resistance with a value of 3.8×10⁻³/° C. The performance of the FGA-based E-skin was evaluated across a broad temperature range from 25 to 62° C. using a commercial hot plate as a temperature controller. As shown in FIG. 15, the resistance changes were highly correlated with the corresponding temperature changes. Additionally, the time response of the FGA-based E-skin to the temperature was fast (a few seconds) upon heating and subsequent cooling (FIG. 16). Note that the performance is limited by the ramp rate of the hot plate. These results indicate that FGA-based E-skin can reliably monitor skin temperature changes in response to environmental stimuli.

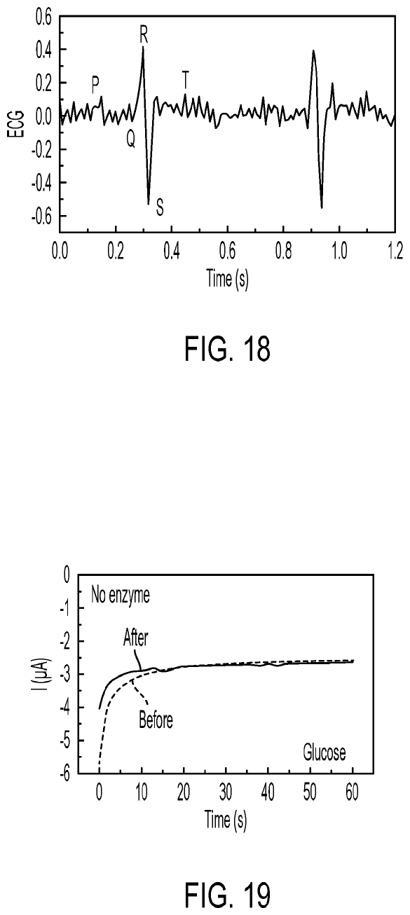

For ECG monitoring, we employed a concentric ring design, enabling Laplacian filtering to localize the potential bioelectric source with spatial resolution regulated by the ring distance. The Ag/AgCl traces were printed to form robust conductive interconnections with a resistivity of a few Q. When mounted on the forearm, continuous ECG measurement is shown in FIG. 17, and the ECG is recorded with identified P, Q, R, and S waveforms depicted in FIG. 18.

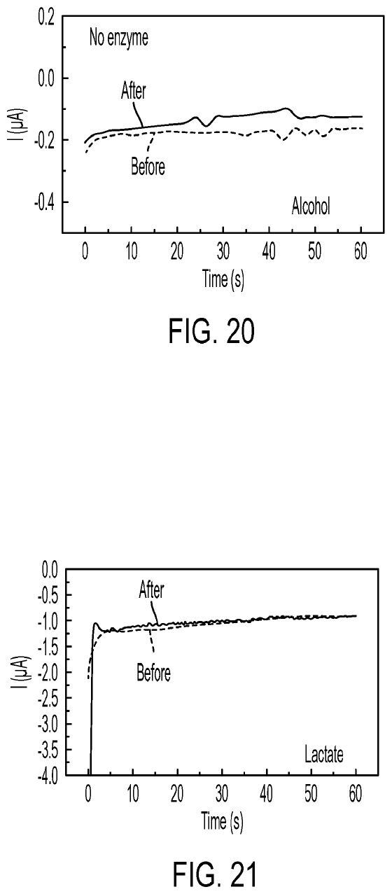

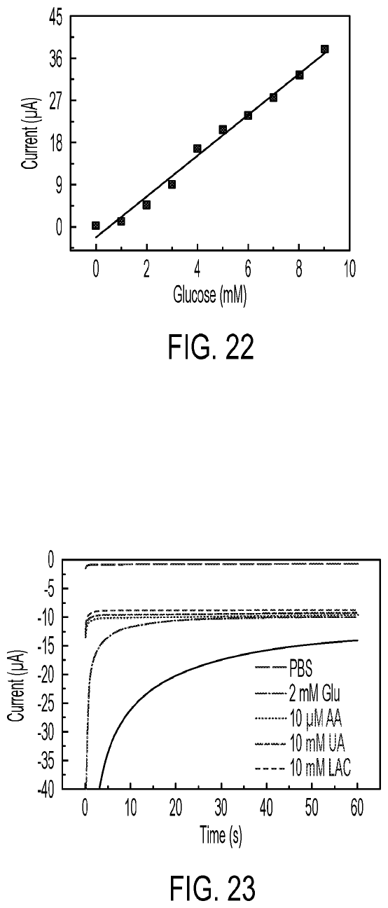

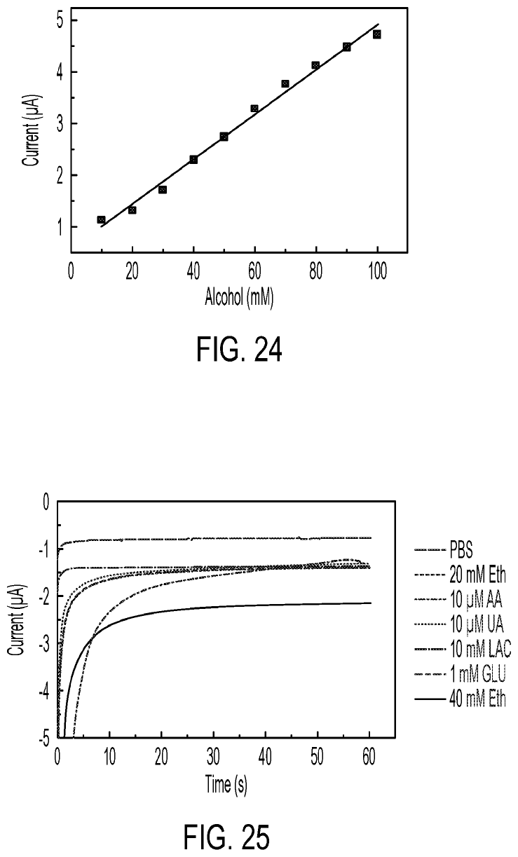

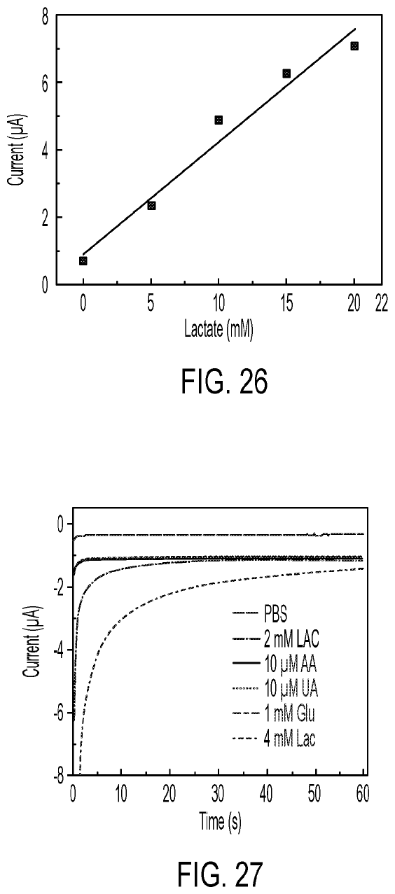

For metabolic biomarker analyses, electrochemical detection was used by implementing non-invasive sweat stimulation via transdermal pilocarpine delivery at the iontophoresis anode and ISF extraction via reverse iontophoresis at the cathode. Alcohol and lactate levels were tracked in sweat, whereas glucose levels were monitored in the ISF by modifying the working electrodes at their respective anodic/cathodic compartments. Chronoamperometry was applied for electrochemical measurements of the hydrogen peroxide generated from the alcohol oxidase (AOx), lactate oxidase (LOx), and glucose oxidase (GOx) enzymatic reactions. By contrast, in control experiments, no signal was captured or detected with the sensors without the surface modification with GOx, LOx, or AOx (FIGS. 19-21). The enzymatic biochemical sensors for glucose, lactate, and alcohol were evaluated by making standard additions to their targets in 0.1 M phosphate-buffered saline (PBS). The amperometric response to glucose was investigated from 0-9 mM with 1 mM increments and showed a strong linear response induced by hydrogen peroxide reduction (FIG. 22). This response was also robust in the presence of electroactive interferents (i.e., ascorbic acid and uric acid) and the other target biomarkers (lactate or alcohol) due to the selectivity of the enzyme-based biosensing (FIG. 23). Similarly, the amperometric response of the alcohol sensor was evaluated over a broader range of 0-100 mM with 10 mM increments (FIG. 24) and showed good selectivity in the presence of physiologically relevant interferents (FIG. 25). Finally, the lactate sensor also showed a robust amperometric response in the tested range of 0-20 mM with 5 mM additions, even in the presence of interferents (FIGS. 26-27).

### Example 3. Flexible Aerogel Synthesis and Characterization

The porous structures are essential to make the GelMA aerogel flexible. Currently, several attempts have been made to regulate pore size and interconnectivity of the GelMA gels by tuning GelMA concentration, photoinitiator concentration, degree of methacrylate substitution, and crosslinking time. A few reports have also explored the effects of freezing temperatures and freezing time on their pore morphologies. It was found that such polymerized GelMA hydrogel with smaller pores could be obtained by lowering the freezing temperatures (i.e., −10 to −80° C.). However, GelMA molecules were covalently bonded together in this case, and the fixed GelMA polymer chain could prevent water molecules from rearranging and impede ice crystal nucleation, yielding low pore interconnectivity and lack of flexibility in the structure. To this end, Mooney et al, studied the effect of subzero temperature (−12° C.) regime during polymerization of the GelMA to develop shape-recovery cryogels with highly interconnected porosity. However, Mooney et al., did not study the impact of their fabrication process on flexibility of the dry cryogels. Indeed, their fabrication strategy could not maintain shape recovery property of the cryogels, when GelMA concentration increased above 1.5% w/v.

Motivated by these previously reported literatures that validate the impact of low-temperature using liquid nitrogen on flexibility of other polymers (i.e., poly(2-hydroxyethyl methacrylate) cryogels, alginate (or cellulose)-based cryogels, and cellulose-chitosan aerogels), we firstly employed such liquid nitrogen-assisted strategy to make GelMA aerogel flexible, which has not been studied before.

In addition, the freezing regime is critical in managing pore size and pore interconnectivity. At sub-zero temperature, water solvent forms ice crystals, acting as inherent porogens. Subsequent freeze-drying removes the ice crystals and maintain the pore structures where the ice crystals had formed. Utilizing these ice crystals as a temperature to regulate porosity, pore size and interconnectivity, we firstly explored the effect of ultra-low temperature regime on the flexibility of GelMA aerogels.

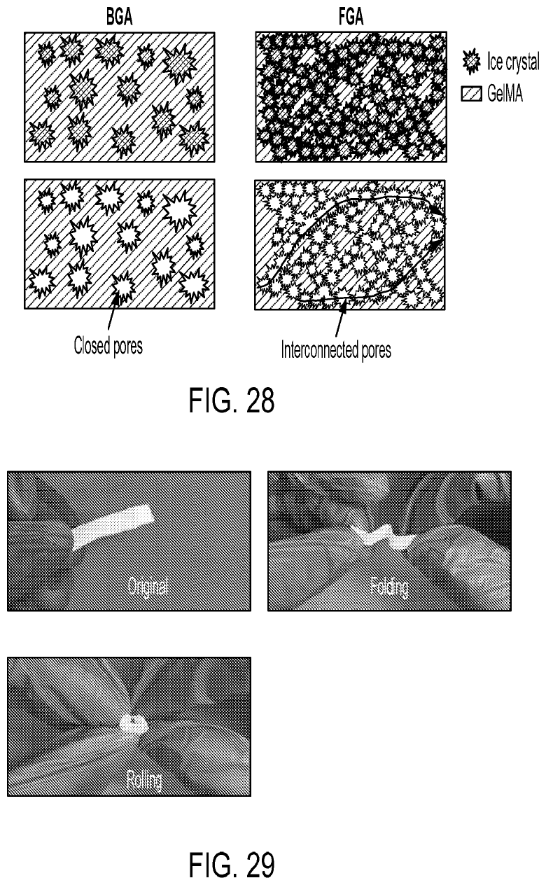

For typical fabrication, GelMA pre-polymer solution was cast in PDMS molds, followed by gelation at room temperature, storage at −80° C. and subsequent freeze-drying, which resulted in the formation of a brittle GelMA aerogel (BGA). Such brittleness could be attributed to the development of irregular ice crystals that randomly formed between the tightly bonded GelMA polymers, resulting in a non-uniform pore structures with low interconnectivity (i.e., in the form of closed pores) as summarized in FIG. 28, left panel.

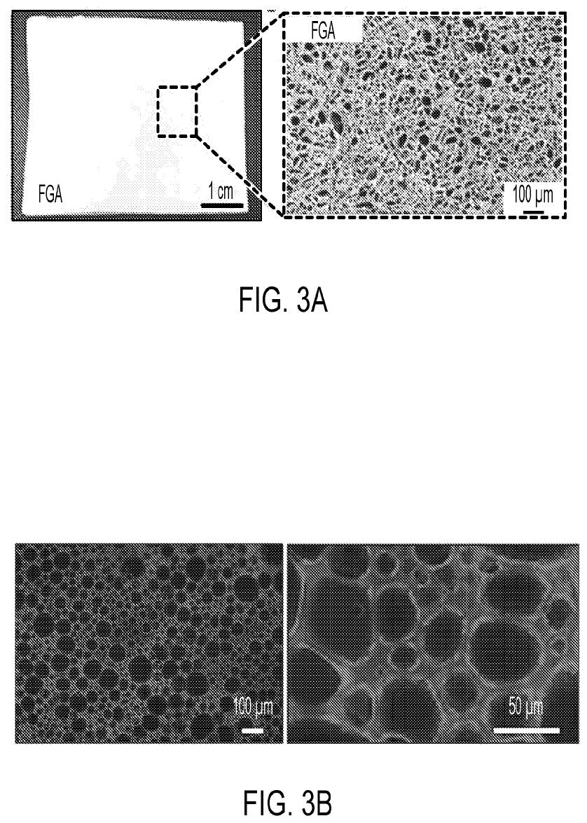

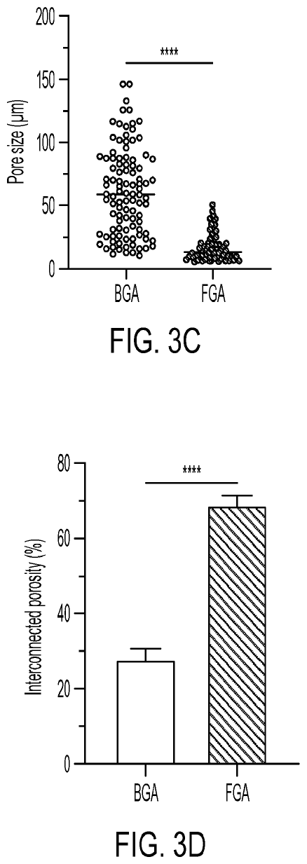

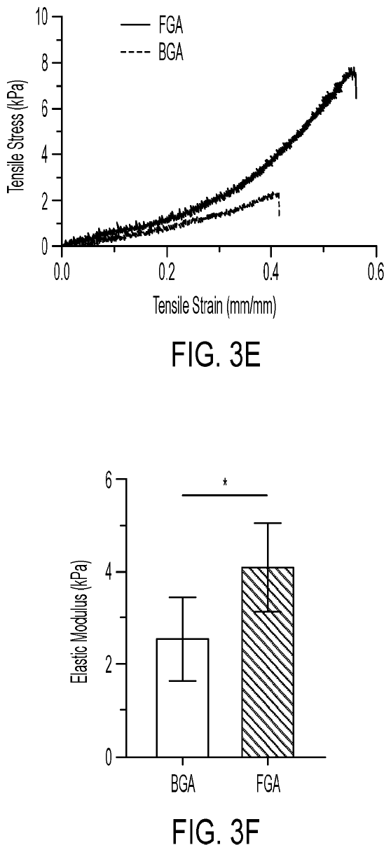

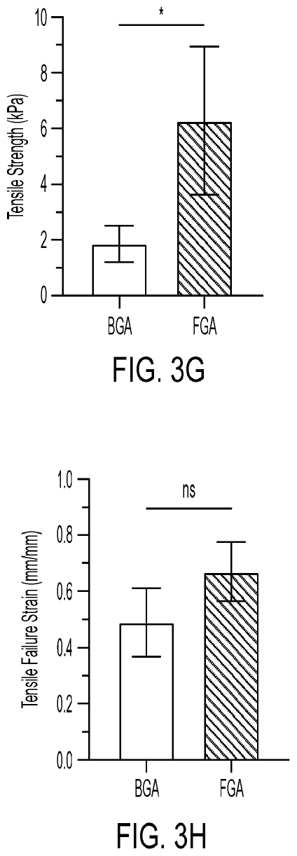

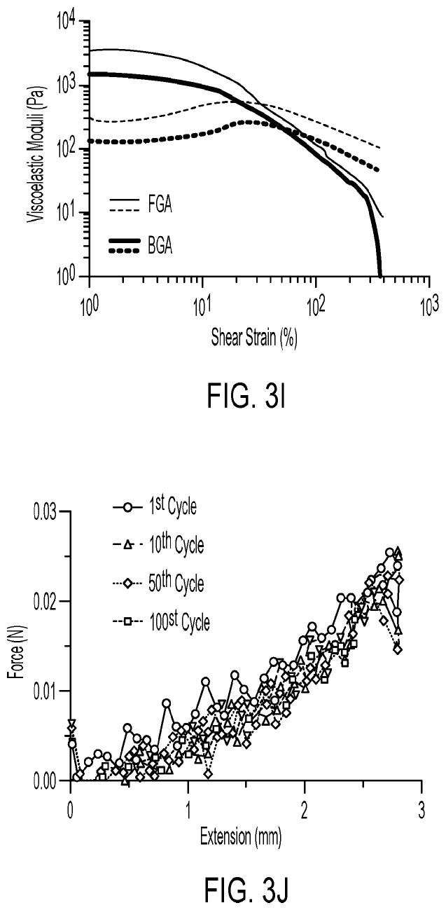

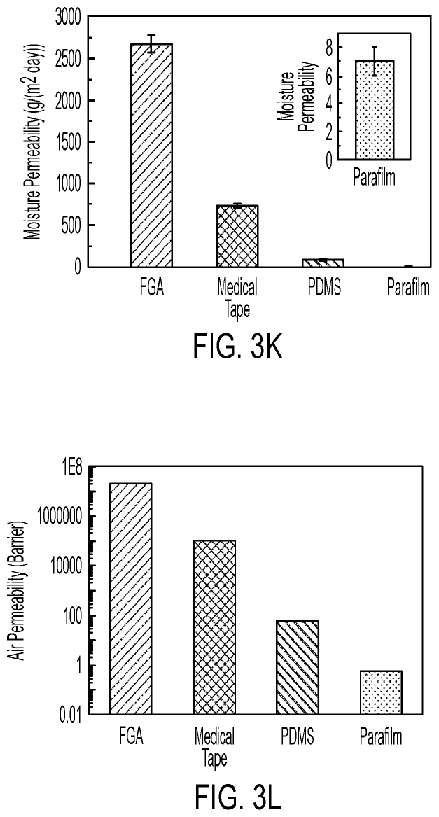

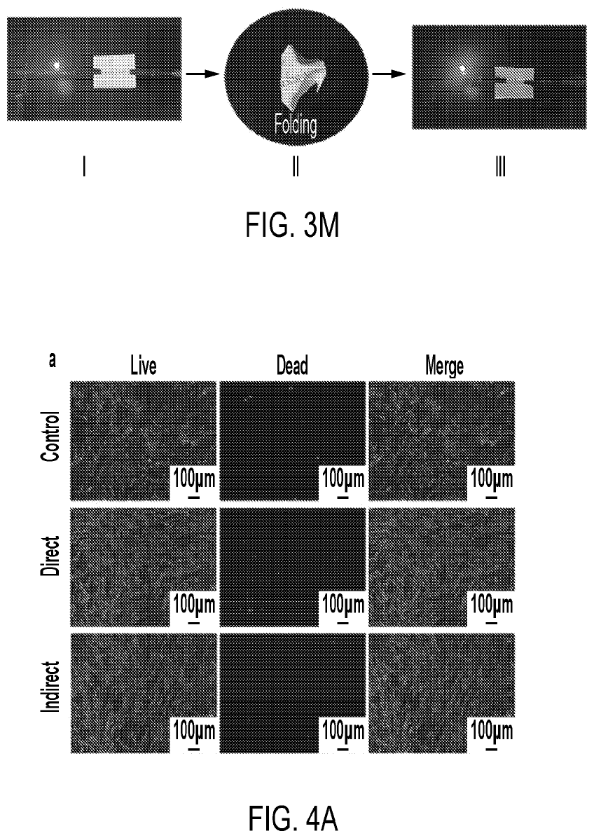

One strategy to offer flexibility to cryostructures is applying freezing temperature before and/or during the polymerization of GelMA chains. In this case, due to the mobility of the GelMA macromolecules, water molecules could freely contact each other and therefore form a unform interconnected ice crystals (FIG. 28, right panel). GelMA would slowly self-organize and polymerize surrounding the ice crystals. Herein, we found that snap freezing of GelMA pre-polymer in liquid nitrogen (ca. −196° C.), followed by slow polymerization at low temperature (−80° C.), and subsequent freeze-drying, yielded flexible GelMA aerogels (FIG. 3A), which easily bend, fold, and roll without breakage (FIG. 29).

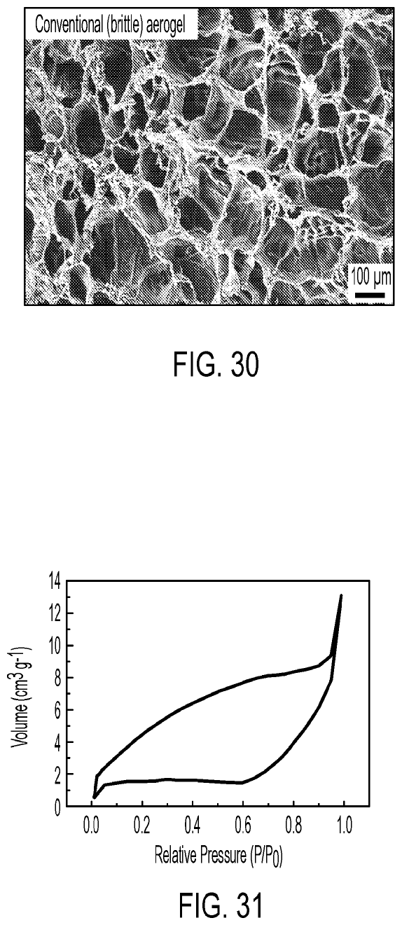

As noted earlier, the freezing temperature has a significant effect on pore structure and morphology. For instance, lower freezing temperature generate smaller pores with higher interconnectivity, because the solvent freezes faster, preventing further growth of ice crystals. To investigate the effects of the fabrication process on the pore size and morphology, scanning electron microscopy (SEM) was performed to show that FGAs have highly porous structures with smaller pore sizes and more uniform pore distributions than BGAs (FIG. 3B, FIG. 30). The average pore size measured from the SEM images was approximately 17 μm and 61 μm for FGAs and BGAs, respectively (FIG. 3C). Since the formation of pores depends on the growth of ice crystals, uniformity in pore size of the FGAs is attributed to cryogenic temperature regime as compared to other sub-zero temperatures (−1 to −20° C.). Furthermore, cryogenic temperatures significantly reduce chemical crosslinking (i.e., covalent interactions) of the methacrylate groups governed by ammonium persulfate (APS) and tetramethylethylenediamine (TEMED), allowing the pronounced effect of physical crosslinking and easy molecular rearrangements that results in smaller and highly interconnected pores in FGAs. Incorporating inorganic salts such as PBS, APS and TEMED in GelMA solutions further enhances physical crosslinking between the polymer chains due to ionic interactions and hydrogen bonding at cryogenic temperatures. Other factors that affect pore structure and flexibility of the cryostructures are concentration of the polymers and thickness of the cryostructures. Higher concentrations of the polymer decrease the porosity, thus reduce the flexibility. The thinner aerogel reduces heterogeneity of the temperature across its depth, resulting in uniform ice crystal formation and better flexibility after freeze-drying.

We further investigated the interconnectivity of the micropores. The FGAs showed interconnective porosity of about 69%, 2.5 times higher than that for BGAs (FIG. 3D). Such highly uniform pore interconnectivity of FGAs supported complete shape recovery of the aerogels after deformation (Supplementary Mov. 1), whereas the BGAs broke under high strain and could not retain their original architectures (Supplementary Mov. 2). Retention of structural architecture for FGAs during mechanical deformation is also attributed to the small pore sizes, which could reinforce increased pore density throughout the aerogel and provide structural support to maintain the framework.

Further characterization of FGAs by nitrogen adsorption analyses showed a pore volume of 0.02 cm³/g with a surface area of 40 m²/g (FIG. 31). FIG. 3E shows representative tensile stress-strain curves of the FGAs and BGAs. The elastic moduli of FGAs calculated from the slope of the stress-strain curves were significantly higher than that of the BGAs (FIG. 3F). The tensile strength of FGAs is approximately 6.3 kPa, which is more than three-fold stronger than BGAs (FIG. 3G). The FGA displays over 70% stretchability compared with BGA (FIG. 3H). The advantageous mechanical properties of FGAs are the result of such highly porous structures and self-organization-mediated physical crosslinking, which provide increased crystallinity in the polymeric network (i.e., hydrogen bonding) at cryogenic temperatures. Similarly, the rheology data demonstrated that FGAs have even higher viscoelastic moduli than BGAs (FIG. 3I), which suggests the presence of extended crosslinked polymer backbones. To characterize the strain recoverability of the FGAs, we performed cyclic tensile tests up to 100 loading and unloading cycles (FIG. 3J). The FGAs could recover almost entirely without significant hysteretic loss, indicating complete energy recovery during unloading. Such recovery during cyclic deformations is related to the presence of noncovalent interactions, which allow the reversible dynamic rearrangement of the physically crosslinked polymeric backbone.

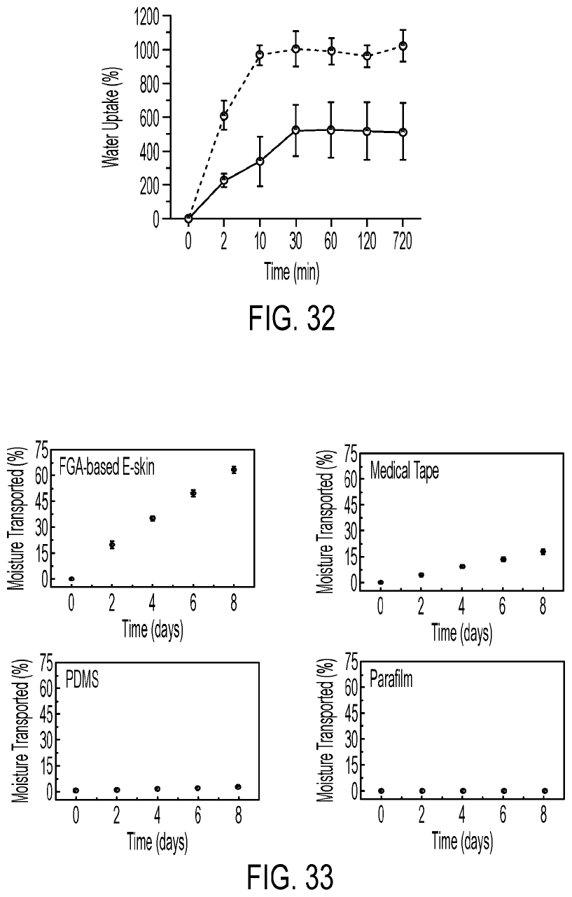

Additionally, the FGAs absorbed almost 1000% of their volume of water within the first 10 min, which is 13 times their original weight, and subsequently reached equilibrium. By contrast, the BGAs absorbed approximately 50% less water than the FGAs and reached equilibrium in 30 min, slower than FGAs (FIG. 32). Note that no obvious changes in the original dimensions and structures of the FGAs were observed after water uptake, indicating excellent anti-swelling behavior, which is essential for FGA-based E-skins.

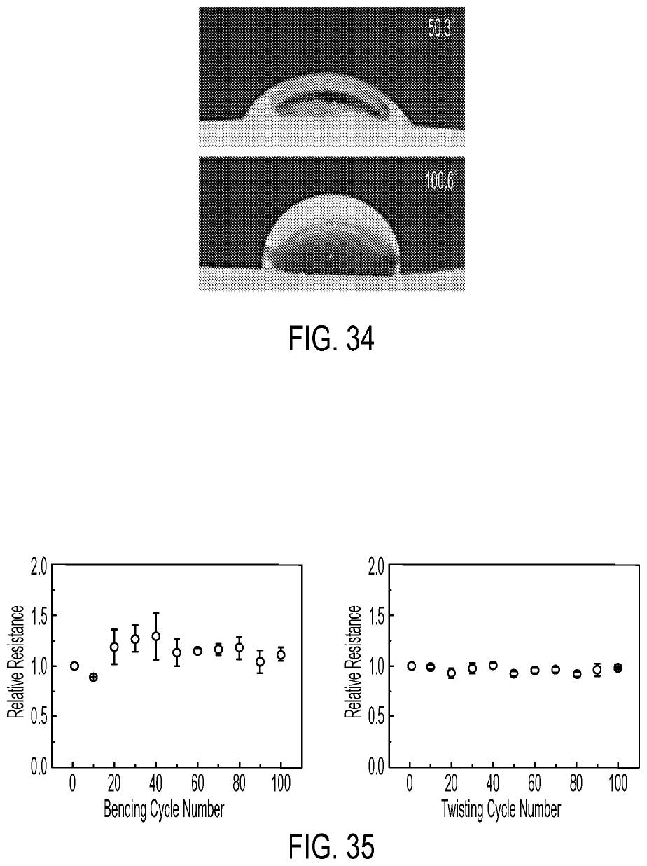

As validated in FIGS. 3K-3L, and FIG. 33, such porous FGAs provide excellent breathability. The moisture permeability of FGAs (2675 g m⁻² day) was much larger than that of PDMS (91 g m⁻² day), Parafilm (7 g m² day), and commercial medical tape (734 g m⁻² day). By contrast, the moisture permeability of PDMS (300 μm), a widely used polymer elastomer in flexible electronics (e.g., electronic skin), was negligible compared to the FGA. At this point, FGAs are hydrophobic. That is, they are permeable to moisture but waterproof. The hydrated FGAs rendered the sample hydrophilic, where the water contact angle of the FGAs was decreased from 100.6° to 50.3° after hydration (FIG. 34), which facilitates efficient contact of the sweat with the sensors. Furthermore, the air permeability of the FGAs was much higher than that of commercial medical tape (over 100 times), which is compatible with the perspiration of the human body under different scenarios such as exercise. In contrast, the air permeability of PDMS and parafilm were below 60 Barrer.

We screen-printed electrodes onto the FGAs to test their mechanical properties under extreme conditions. After crushing and then unfolding the electrodes on FGA, the electrodes remained conductive, as shown in FIG. 3M. The mechanistic explanation is that soft conductive ink printed on this FGA substrate remains conductive during folding and unfolding, undergoing deformation to tolerate the applied stress. To test the robustness of the printed electrodes on the FGA, we investigated cyclic bending and twisting deformation while monitoring the resistance changes across the electrodes. As shown in FIG. 35, the relative resistance typically varied less than 20% (with initial resistance values ~50Ω), even after 100 cycles of deformation. Such results confirm that the mechanical flexibility of the FGAs, in combination with stretchable and screen-printable inks, yields robust electrical contacts.

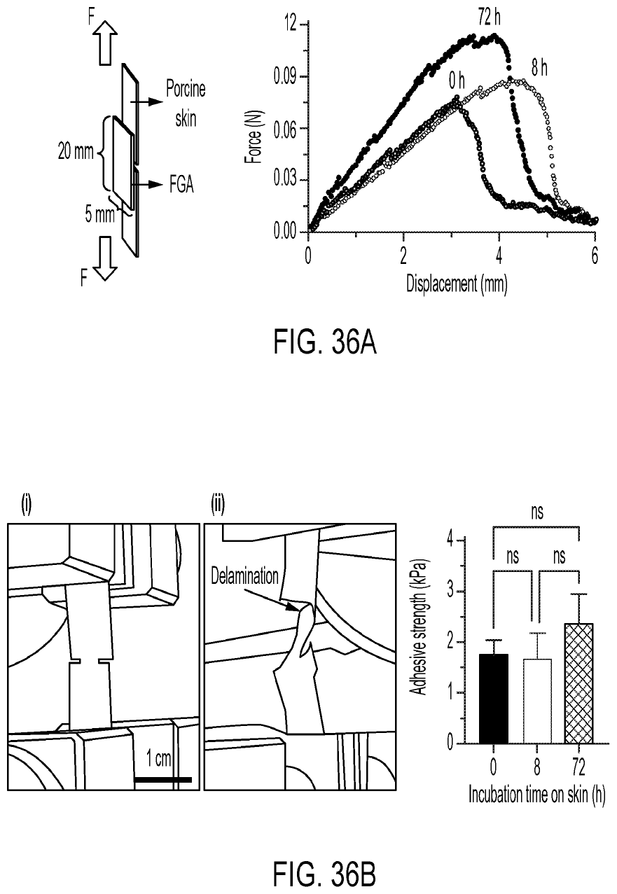

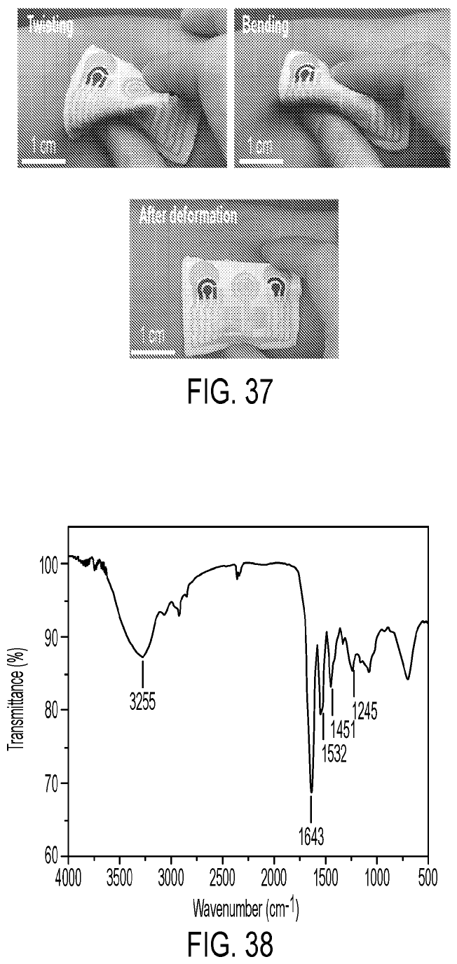

Further, we investigated the direct and conformal contact of the FGA to the skin using a standard wound closure test setup (FIG. 36A). The FGA patch (100 mm²) was applied on porcine skin, where they were incubated for varying amounts of time. The skins were then axially displaced while the adhesion force was recorded. As shown in FIG. 36B, the adhesion force steadily increased by displacement for all the samples until the FGA patch was detached from either side of the skin, corresponding to the abrupt decline in force. At early stages of the test, the interfacial adhesion was strong enough to attach the FGA patch to the dynamic skin tissue (FIG. 36B-I), until the excess load forcefully delaminated the FGA (FIG. 36B-II). Regardless of the time attached on the skin, all the FGA patches showed adhesive strengths over a range of 1.5 to 2.5 kPa (FIG. 36B), which is consistent with the reported values for adhesive patches. We attribute the self-adhesive property to the rapid absorption of body fluids by dry FGA, facilitating electrostatic forces and hydrogen bonding with the tissue surface. These results make the FGA an attractive candidate for E-skin devices that adhere to the skin. However, to enhance the adhesive strength and to maintain the long-term stability and durability of the FGA patch on the skin, a small amount of the commercial medical tape was applied (only) on the boundaries of the FGA-based E-skin, ensuring that the breathability of the device was not significantly affected by the tape. The twisting and bending of the patch on the skin are shown in FIG. 37, indicating its on-body conformability and mechanical integrity during bending, twisting and after deformation.

### Example 4. Biocompatibility, Non-Inflammatory, and Biodegradability of the Flexible Aerogel

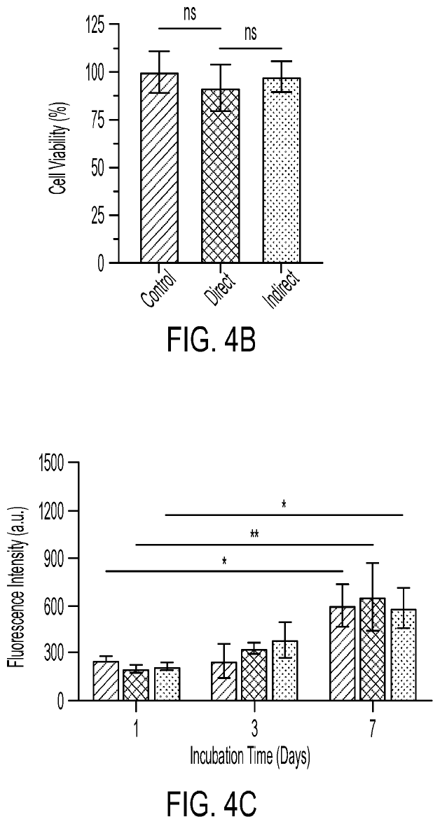

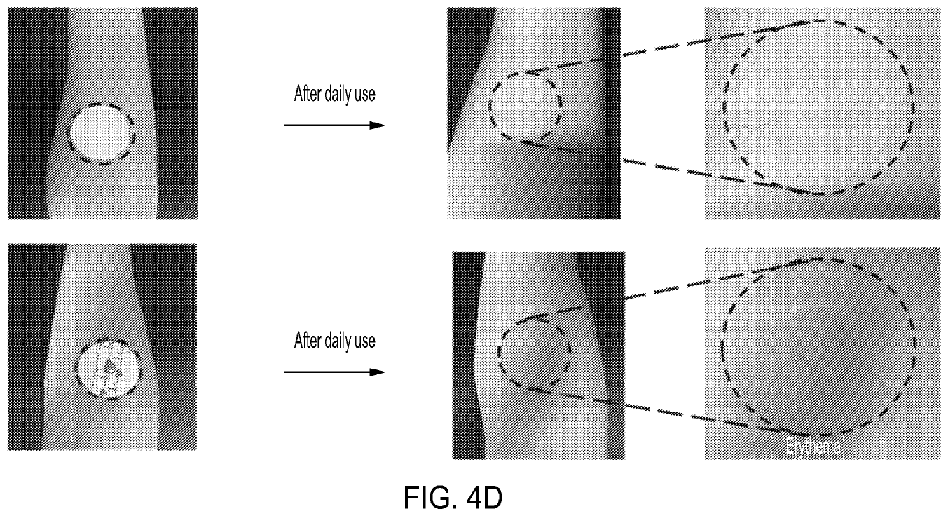

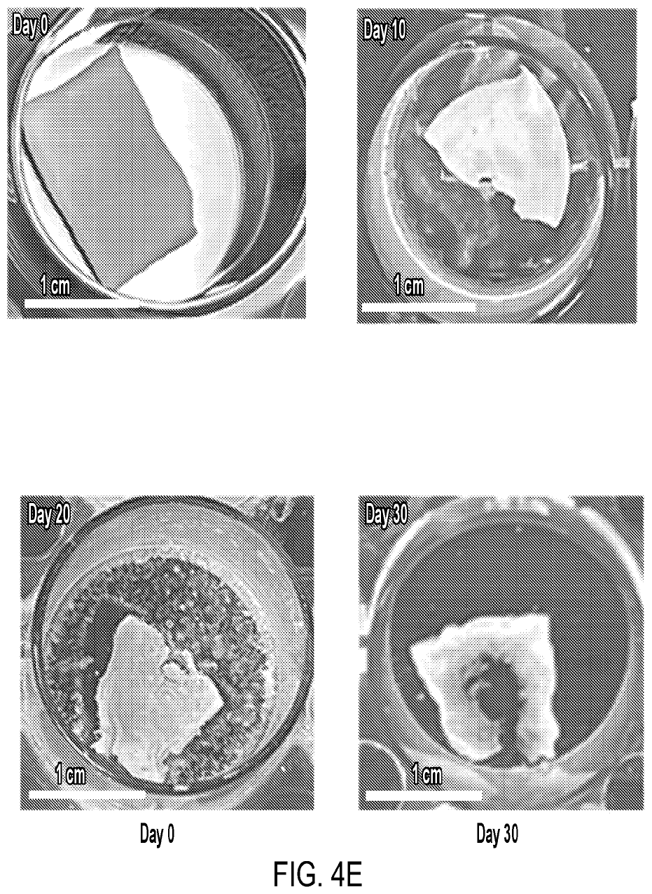

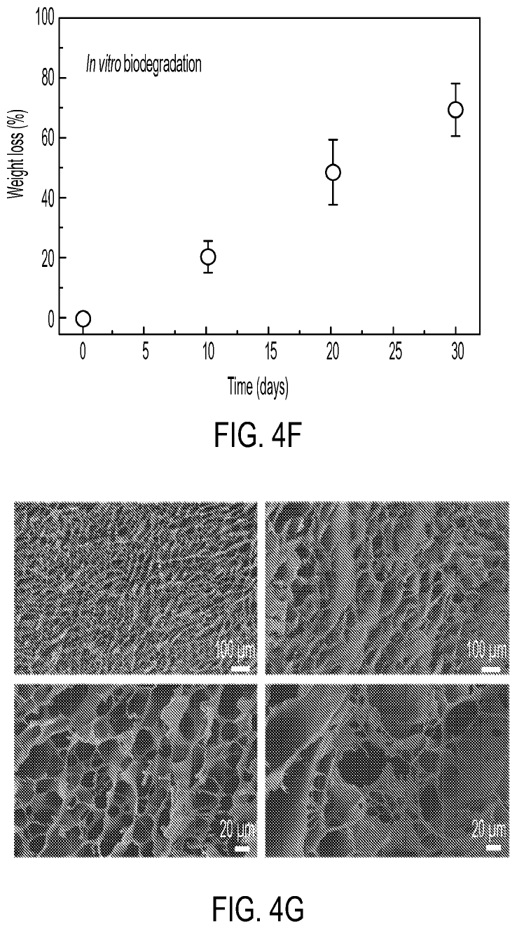

To assess the biocompatibility of the aerogel on human skin, in vitro cytocompatibility assay against human dermal fibroblast (HDF) cells was performed. Direct incubation of the cells on the surface of the FGA-based E-skin did not induce a negative effect on the morphology of the cells for up to 7 days. Staining live cells with green-fluorescent dye (Calcein AM) suggested excellent cell viability, as a majority of the cells stained green (FIG. 4A-I). Likewise, when the FGA-based E-skin was not directly in touch with the cells and placed inside the transwells (indirect method), the cells maintained their elongated morphology with a slightly smaller number of dead cells (red spots in FIG. 4A, I) compared to the direct method. The overall viabilities of the cells in the direct and indirect methods were 91% and 97%, respectively, compared to the non-treated control (FIG. 4A-II). Moreover, extended incubation of the cells with the FGA-based E-skin for up to seven days did not affect the metabolic activity and proliferation of the cells, as indicated by the PrestoBlue assay. The cells in direct and indirect contact with the FGA-based E-skin showed higher fluorescence intensity by day 7 than by day 1, indicating no adverse effects on metabolic activity of the cells (FIG. 4A-III).

We also conducted on-skin tests to validate the importance of breathability for long-term wearability. For comparison, two patches (the FGA-based E-skin and the commercial gel electrode) were attached to the forearms of one adult subject for one day of use. As illustrated in FIG. 4B, the breathable FGA-based E-skin did not cause any adverse effects on the skin after daily use. In contrast, the commercial gel electrode induced obvious signs of skin erythema because of its poor permeability.

Biodegradation represents a significant feature of the E-skin, enabling it to operate over the desired time frame while still physically degrading into nonharmful constituents after use. GelMA is well-known to be biodegradable and can successfully break down over a controlled period. We carried out in vitro biodegradation investigations of FGA-based E-skin over 30 days. The variations of their morphology and weight loss after corresponding degradation periods were recorded (FIG. 4C-I). The results revealed bulk degradation and autocatalytic hydrolysis after 20 days of incubation, with weight loss of almost 60% (FIG. 4C-II). Moreover, it reduced at least 80% of its original weight a 30-day degradation period. Concurrently, cracks appear on the FGA-based E-skin surface, and its density increases as the degradation progress (FIG. 4C-III). Additionally, the degradation time of the FGA-based E-skin is tunable based on the thickness of the GelMA aerogel and concentration of GelMA. It can also be tailored depending on the intended wearable lifetime of the device.

### Example 5. Passive-Cooling Capabilities of the Flexible Aerogel

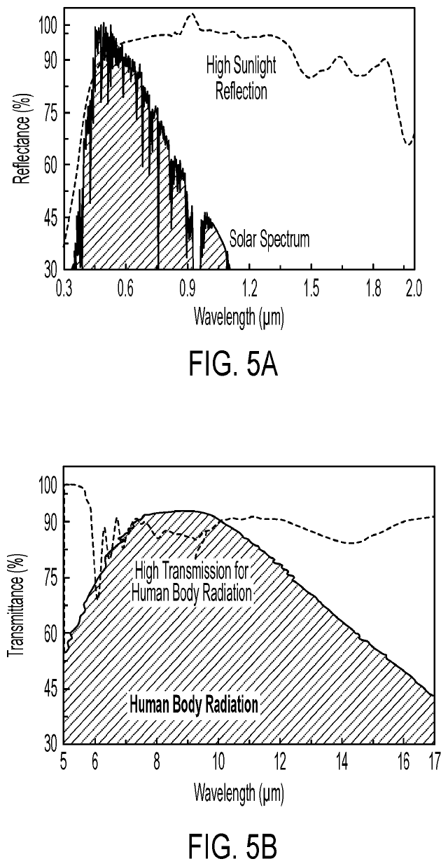

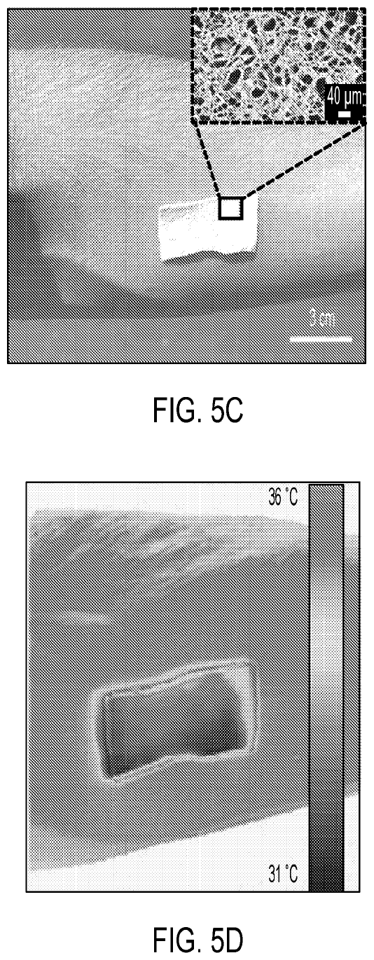

Point-of-care wearable thermal management strategies to address Joule heating and personal cooling can help regulate human body temperature, significantly improving user comfort and energy saving. Although E-skins with personalized heating capabilities have been widely investigated, Developing E-skin that can passively cool human bodies without external energy consumption remains less explored. Porous materials, such as polyethylene, poly(vinylidene fluoride-co-hexafluoropropene), and polystyrene-blockpoly (ethylene-ran-butylene)-block-polystyrene, have been reported with passive-cooling features. However, the balance between pore-size control and skin-similar mechanical properties impedes their ability to be integrated with E-skin platforms. Considering the importance of the thermal management for E-skin applications, which could counteract deleterious phenomena, such as Joule heating, to improve human comfort and to reduce energy consumption, we also investigated the potential passive-cooling effects of the FGA-based E-skin. FIG. 5A and FIG. 5B depict the Fourier-transform infrared (FTIR) and ultraviolet-visible (UV-vis) spectra of the FGA-based E-skin, respectively. Due to the multiscale porous nature of the flexible GelMA aerogel, it can effectively reflect sunlight while allowing human-body mid-infrared to be transmitted largely unimpeded. The spectrum of the GelMA can be characterized by comparing the characteristic peaks at 1532 cm⁻¹ and 1643 cm⁻¹ in FIG. 38, which are attributed to the C—N stretching of amide groups and CO stretching of the gelatin peptide groups, respectively. Additional peaks for GelMA were reported by Arica et al. and include 3255 cm⁻¹, 1451 cm⁻¹, and 1245 cm⁻¹ which were attributed to N—H stretching, and in plane stretching of —CH₂ groups, respectively. Given these promising results, we conducted an outdoor on-body test by wearing the FGA on the skin (FIG. 5C). The inset reveals its highly porous structure. A thermal camera was employed to compare skin temperature with/without the FGA-based E-skin. These results show that the temperature of the skin covered with the FGA was maintained at temperatures ca. 5° C. lower than that of uncovered skin after 30 min of sunlight radiation exposure (FIG. 5D).

### Example 6. On-Body Validation of the Flexible Aerogel-Based E-Skin with Multiple Stimuli

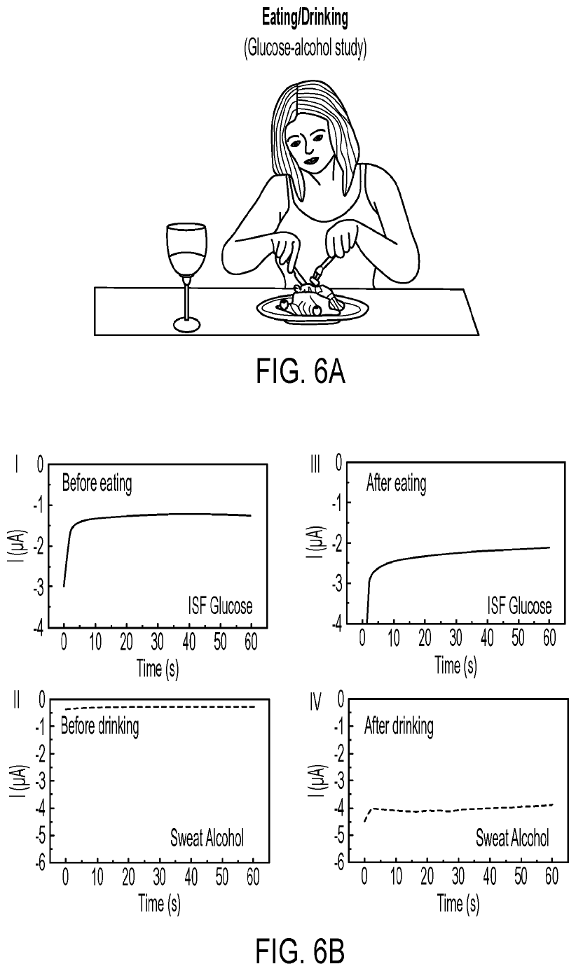

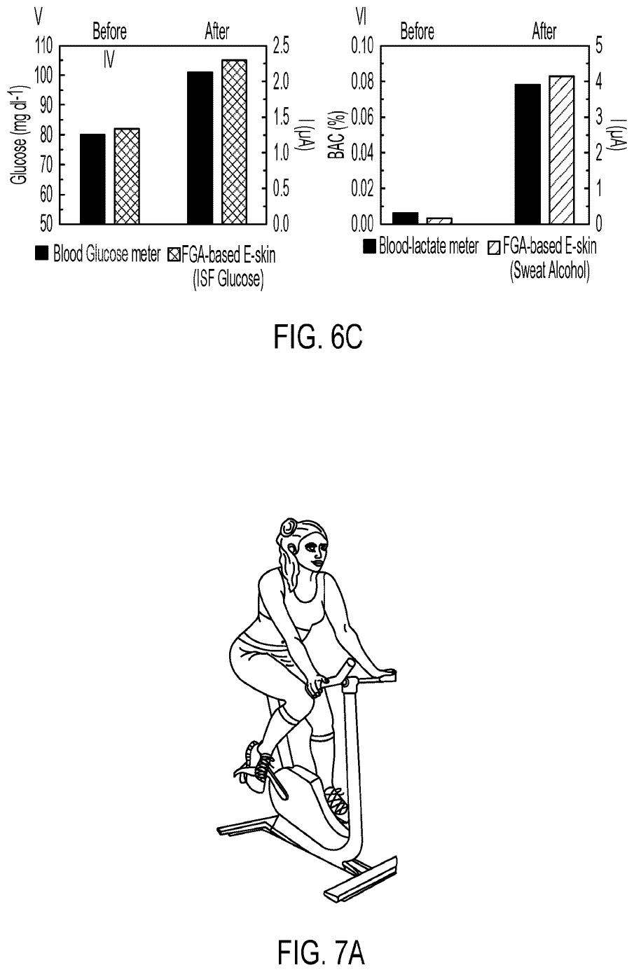

We then validated the E-skin's performance in realistic scenarios in which subjects underwent various activities or were exposed to multiple stimuli that may have competing effects on the physical, physiological, and/or metabolic responses of the body. The E-skin was first investigated for use in tracking physical and physiological signals in addition to multiplexed biochemical monitoring on subjects exposed to different stimuli. A typical paradigm to demonstrate a direct effect on glucose and alcohol levels is consuming sugar- and alcohol-containing aliments or participating in an exercise routine. The reasoning is that glucose levels can be quickly depleted during exercise to produce energy, while glucose/alcohol can be rapidly metabolized while eating/drinking. To study these effects, the subject was asked to have a sugar-rich meal with alcohol-containing red wine while the ISF glucose and sweat alcohol levels were tracked before and after each step. Normal glucose and alcohol levels were observed before eating/drinking (FIGS. 6A-6C-I, II). Afterward, the glucose and alcohol increased accordingly (FIGS. 6A-6C-III, IV). For comparison, a commercial fingerpick blood glucose tester and breathalyzer were applied to measure the blood glucose and blood alcohol levels separately, which have similar trends as the FGA-based E-skin (FIGS. 6A-6C-V, VI). These results indicate that the FGA-based E-skin is able to detect and track daily activities, including simultaneous food and wine intake, the digestion of food and wine that produces glucose as an energy reservoir, and the accumulation of alcohol in the blood. Excessive alcohol consumption can cause severe hypoglycemia and hypotension, even when administered simultaneously with glucose intake, especially for insulin-dependent diabetics. Therefore, concurrent monitoring of alcohol and glucose is helpful in differentiating between moderate and excessive drinking and can help prevent drinking-related incidents, particularly for those with underlying health issues.

Exercise, which includes any physically demanding action, has a significant impact on the physical, physiological, and metabolic response of the body, including changes in body temperature, skin hydration, ECG patterns, heart rate, and glucose/lactate levels. During high-intensity exercise, the lactate levels in the blood and sweat increase because of the anaerobic metabolic pathway, whereas blood glucose levels decrease due to glucose consumption during exercise to produce energy via aerobic metabolism. Meanwhile, the heart rate rises to meet the muscles' demand for oxygen, and the average body temperature increases according to sharply increased muscle metabolism. Concurrently, the skin hydration level also increases due to sweat generation. To study these effects, the subjects were asked to have a sugar-rich meal and then to perform high-intensity stationary cycling for 30 min, followed by 5 min of rest.

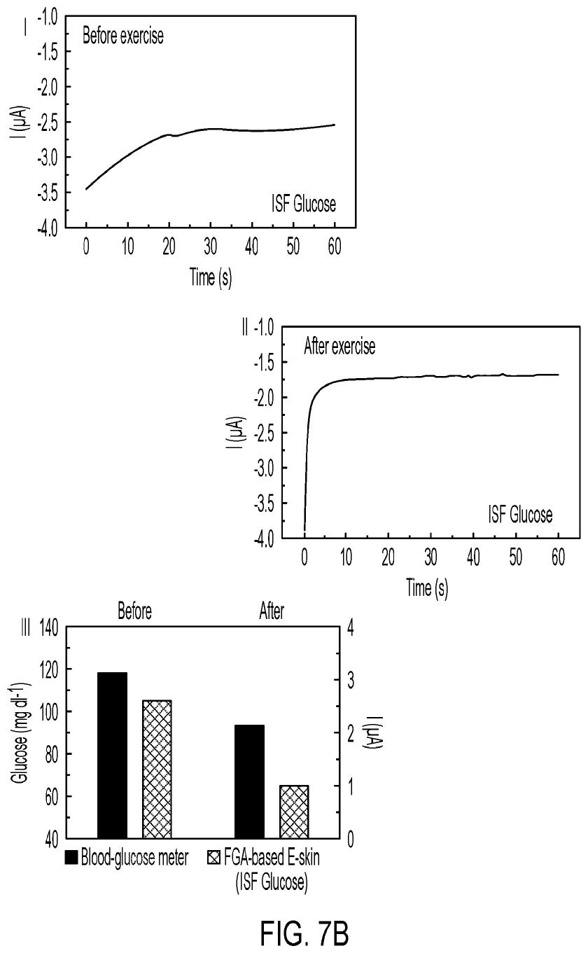

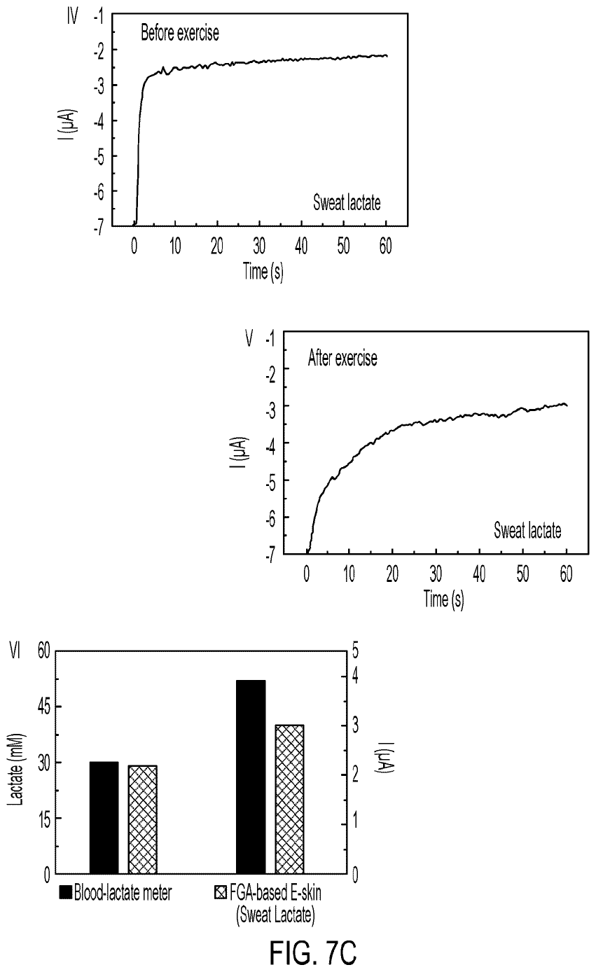

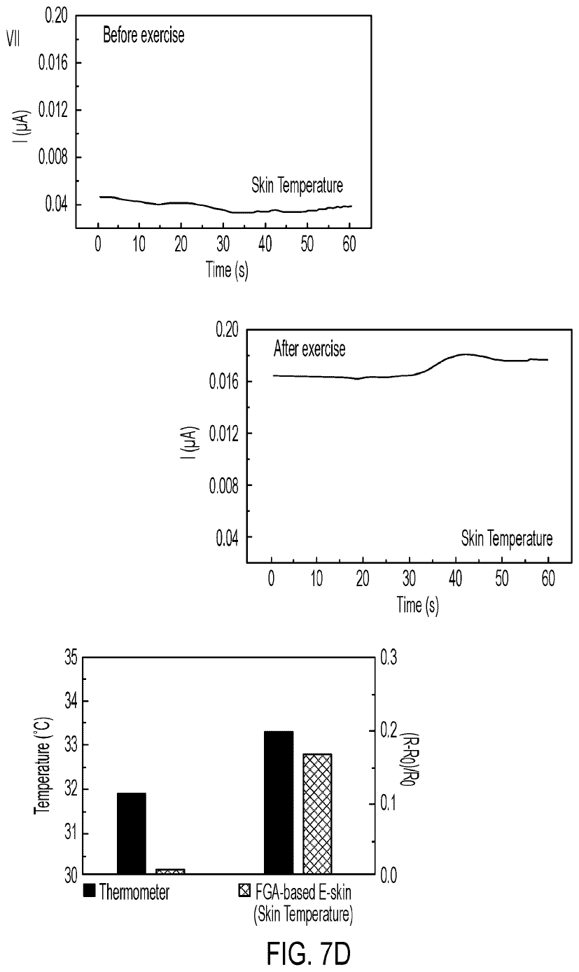

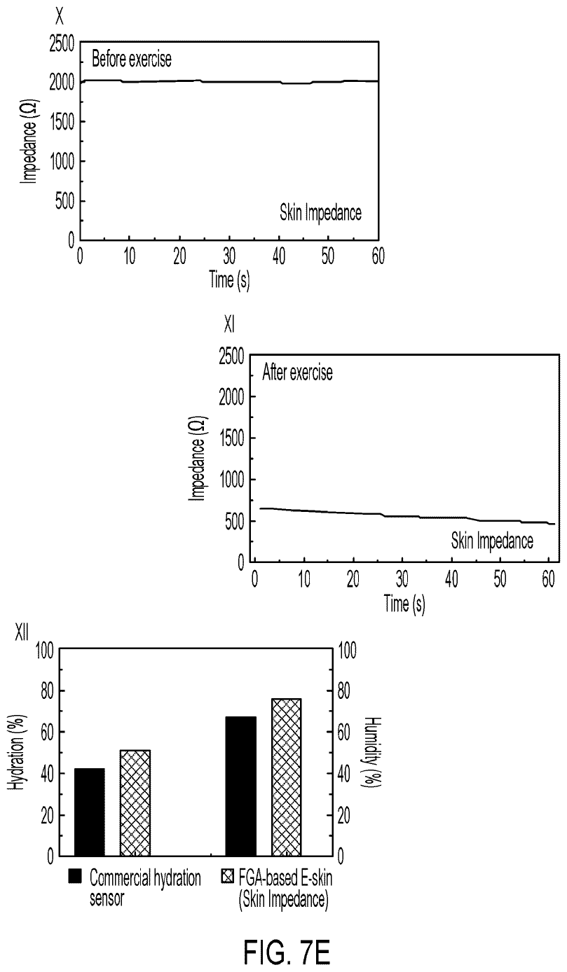

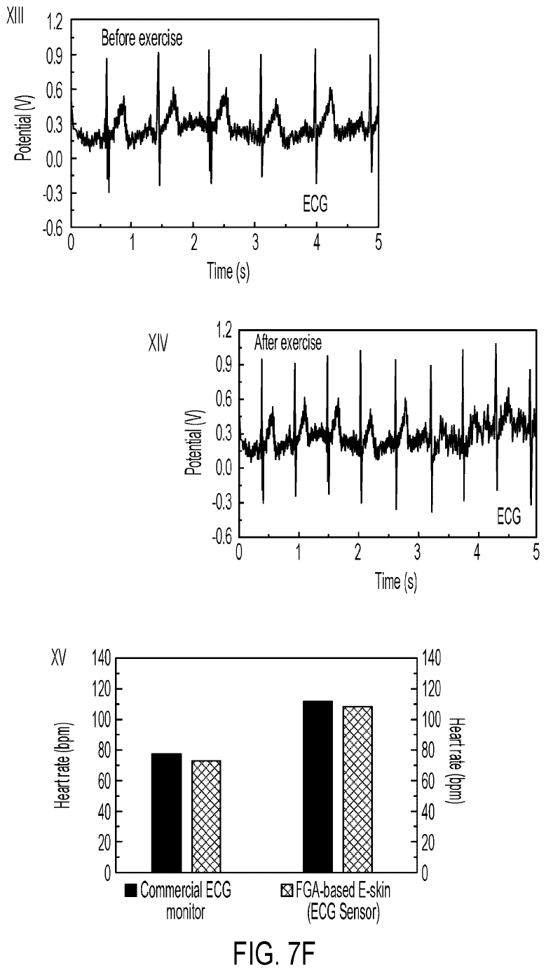

The ISF glucose, sweat lactate, skin temperature, skin impedance/hydration levels, and ECG patterns were monitored by FGA-based E-skin before and after exercise. As shown in FIGS. 6A-6C, high glucose levels (FIGS. 7A-7F-I), low lactate levels (FIGS. 7A-7F-II), normal skin temperature (FIGS. 7A-7F-III), impedance/hydration (FIGS. 7A-7F-IV), and ECG patterns (FIGS. 7A-7F-V) were measured before the cycling activity. The glucose level (FIGS. 7A-7F-VI) and skin impedance decreased after exercise (FIGS. 7A-7F-IX), accompanied by an increase in sweat lactate levels (FIGS. 7A-7F-VII), skin temperature (FIGS. 7A-7F-VIII), and heart rate (FIGS. 7A-7F-X). The findings are consistent with a previously reported study using a similar electrochemical monitoring system. Note that all the sensor performances were validated by commercial devices, as shown in FIGS. 7A-7F-XI,XII,XIII,XIV,XV.

### Example 7. Continuous On-Body Validation of Flexible Aerogel-Based E-Skin

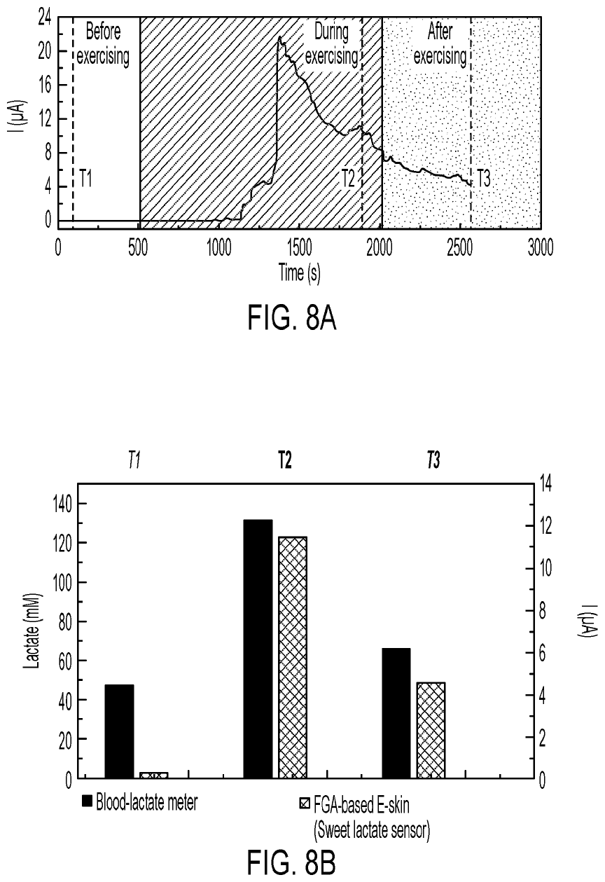

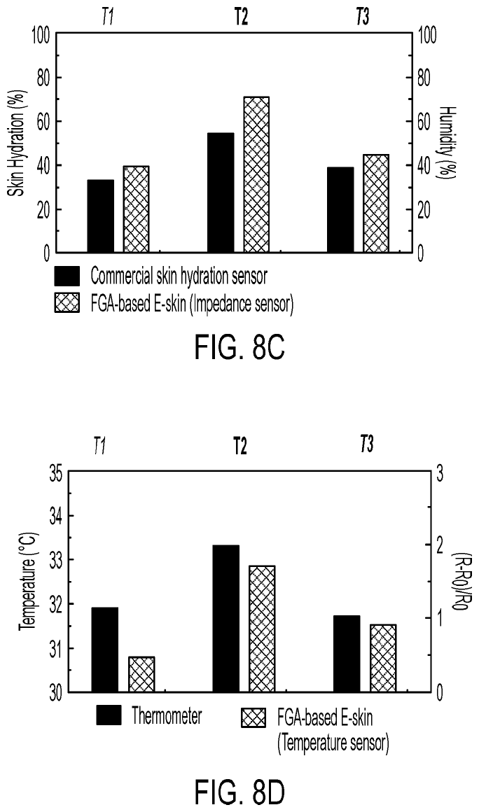

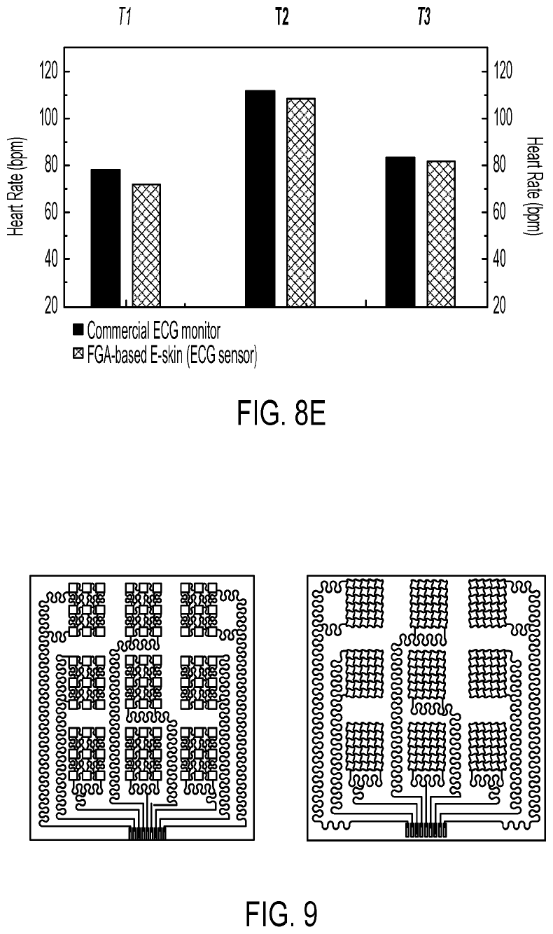

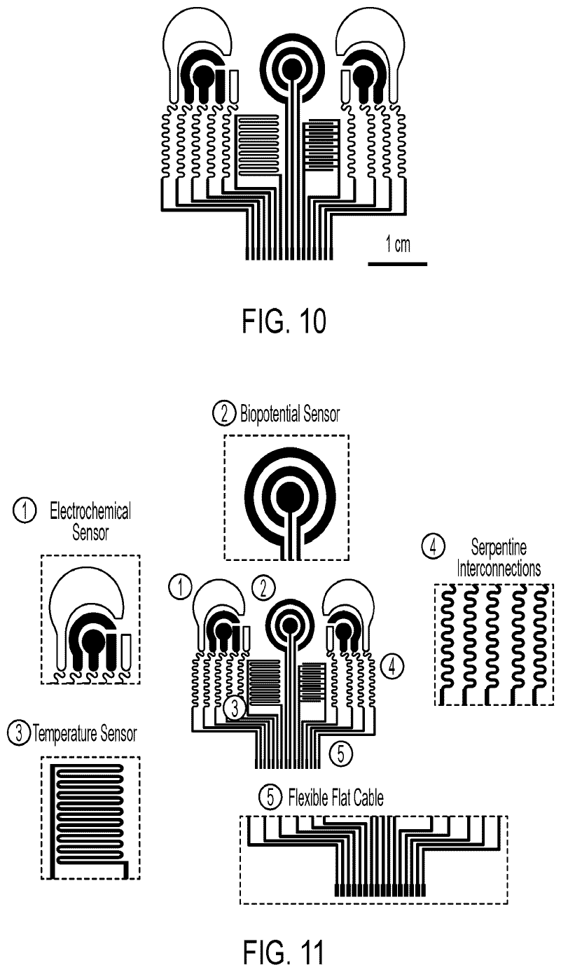

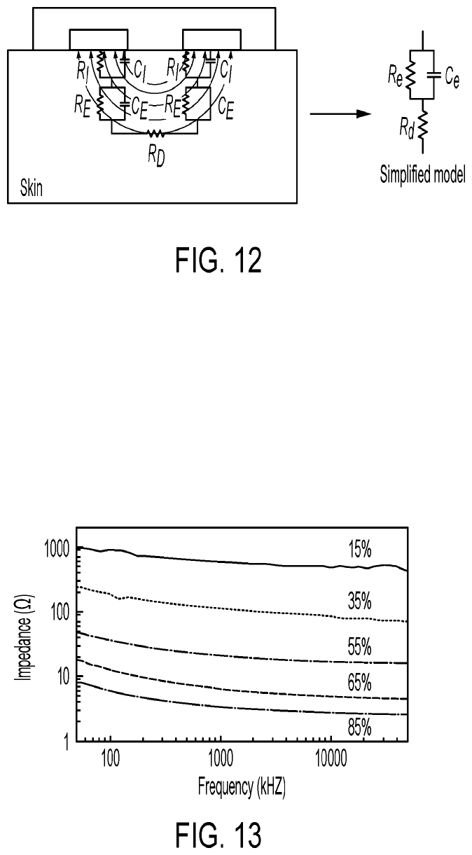

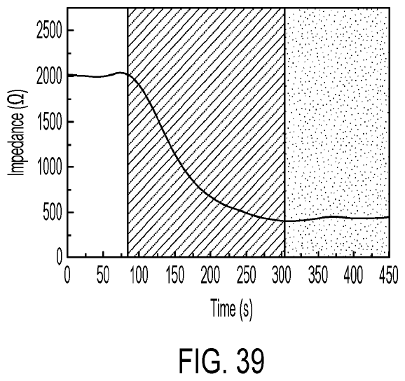

In addition to the previously demonstrated end-point measurements, the FGA-based E-skin is also capable of continuously capturing dynamic biosignals and physiological signal fluctuations in real-time, which was demonstrated in FIGS. 8A-8E and FIG. 39. Physically active individuals are expected to have lower resting heart rates, indicating efficient heart function and good cardiovascular fitness. The lower resting heart rate can be characterized by smaller heart rate increases, as higher cardiac output is required. Smaller increases in lactate levels, temperature, and skin hydration are also expected. The heart rate of an individual is expected to increase following an intense workout, eventually returning to normal baseline levels. Such complex dynamic processes require hybrid sensors (such as electrochemical and physiological sensors) to capture these real-time fluctuations through the activity continuously. In order to validate the ability of the FGA-based E-skin to capture and to detect such changes, subjects were asked to perform high-intensity exercises. The continuous sweat lactate profile was measured by FGA-based E-skin, indicating higher sweat lactate levels during the workout (FIGS. 8A-8B). Additionally, higher hydration levels (FIG. 8C), skin temperature (FIG. 8D), and heart rate (FIG. 8E) were recorded before, during, and after exercise, which had consistent trends. Therefore, it can be concluded that durable and reliable FGAs hold promise in personalized healthcare applications, rehabilitation of patients, and athletic performance optimization.

The seamless integration of biomaterials with diversified functions is a significant breakthrough in developing next-generation flexible electronics, including wearable and implantable electronics. There are two main aspects to these advances: 1) Although current flexible electronics with high sensitivity and high functional integration have been developed, conventional polymeric substrates with mechanical mismatches and limited moisture/gas permeability can cause skin inflammation and irritation and reduce comfort, especially for the long-term on-body use. However, such comfort, safety, and inflammation concerns are often ignored, impeding practical applications to a large extent. GelMA is a promising candidate to address these long-standing issues due to its tunable mechanical properties, excellent biocompatibility, and breathable porous network. 2) The majority of electronic substrate materials are not degradable or recyclable, which introduces plastic waste once they reach their end-of-life period and potentially pollute the environment downstream. GelMA-based biodegradable green electronics can function over the prescribed time frames and then degrade completely into nonharmful products without any adverse effects, alleviating the environmental burden of wearable electronics.

Continuous monitoring of physical, physiological, and metabolic biomarkers can potentially offer new insight into the pathophysiology of the body. This multifunctional GelMA aerogel E-skin platform has thus been validated to support the possibility of developing hybrid wearable sensors with complex integration of physical, physiological, and chemical sensing functionalities on a single flexible and conformal patch to record multiple vital signs in real-time.

A breathable, passive-cooling, biocompatible, biodegradable, and flexible FGA-based E-skin for non-invasive, real-time, and simultaneous monitoring of hybrid chemical-electrophysiological-physical signals has been demonstrated. The performance of this aerogel E-skin was validated by monitoring the ISF glucose, lactate, and alcohol levels from sweat, skin temperature, impedance/hydration, and ECG patterns as model analytes. This multiplexed configuration was investigated in response to stimuli associated with regular daily activities, such as fitness, exercise, and food and wine consumption. Although the integrated aerogel E-skin platform suggests promising functionalities, plenty of opportunities remain to improve such devices: (1) full miniaturization of the system through the development of integrated electronics capable of processing and wirelessly transmitting data from the various sensor modalities; (2) integrating other modules (for example, drug-delivery systems) to form a closed-loop theranostic platform; (3) expanding the multiplexed sensing capability to additional analytes (e.g., growth factors, insulin, or cortisol); and (4) conducting comprehensive validation involving more participants with different health conditions, including patients with cardiovascular disease, diabetes, and/or alcohol use disorder. By addressing these opportunities, an integrated multiplexed wearable healthcare system that can offer insights into the physiological and overall health status of individuals while empowering them to self-regulate and manage various chronic diseases can be envisioned. This work paves the way for a multifunctional aerogel-based E-skin capable of providing informative data regarding human healthcare and lays the foundation for next-generation, patient-centered diagnostic and therapeutic tools.

### Example 8. Mechanism of Impedance Sensing

The mechanism of impedance sensing can be electrically modeled using a series of capacitors and resistors. The contact interface between the electrode and the skin surface can be described by a resistor in parallel with a capacitor, which is dependent on the applied pressure and the humidity of the skin. The epidermis is modeled by a parallel circuit consisting of a capacitor and a resistor. The dermis and underlying subcutaneous tissues, mainly composed of blood vessels, nerves, preparatory glands, and hair follicles, exhibit a pure resistive behavior and can be modeled by a resistor. Due to the symmetry of the two electrodes, the equivalent circuit can be approximated as a parallel-connected resistor and capacitor arising from the electrode-skin contact interface and epidermis, in series with a resistor from the dermis and the underlying tissue. Increasing the water content of the skin increases the conductivity and the dielectric constant of the skin by providing more conductive pathways. The decreased contact impedance and deceased impedance from the epidermis result in decreases of the measured skin impedance as a function of skin hydration level.

### Example 9. Methods and Materials Used in Examples 1-8

Materials: Porcine gelatin, Dulbecco's phosphate-buffered saline (DPBS), Dulbecco's modified eagle medium (DMEM), methacrylic anhydride, dialysis tubing (12-14 kD), Milli-Q water, tetramethylethylenediamine (TEMED), ammonium persulfate (APS) were purchased from Thermo Fisher Scientific (Waltham, MA). SYLGARD silicone elastomer kit was obtained from Dow (Midland, MI). Glucose oxidase (GOx), lactate oxidase (LOx), chitosan, bovine serum albumin (BSA), agarose, pilocarpine nitrate, acetic acid, ascorbic acid (AA), uric acid, ethanol, lactate, glucose, 0.5% trypsin-EDTA, fetal bovine serum, and penicillin/streptomycin (Pen/Strep, 10000 U/mL), kerosene, safflower oil, propanol, and Triton-X-100 were obtained from Sigma-Aldrich (St. Louis, MO). Prussian blue (soluble) and carbon ink were purchased from Sun Chemical Ltd (Parsippany-Troy Hills, NJ). The Ag/AgCl conductive ink was obtained from Creative Materials, Inc (Ayer, MA). The human dermal fibroblast was purchased from American Type Culture Collection (ATCC), USA. All reagents were used without further purification.

Fabrication of gelatin methacryloyl: Gelatin methacryloyl (GelMA) was prepared according to a previously published procedure. The reaction started by adding 10 g of gelatin to 100 mL of pre-heated DPBS on a magnetic stirring hotplate (240 rpm) at ~50° C. Methacrylic anhydride (MA, 8 mL) was then added dropwise to the dissolved gelatin under continuous stirring at dark and allowed to react for ~2 h at 50° C. and 240 rpm. Next, 100 mL of pre-heated (~40° C.) DPBS was added to the solution and magnetically stirred for ~10 min at 50° C. to stop the reaction. The solutions were transferred to dialysis tubing and dialyzed against Milli-Q water at 40° C. and 400 rpm for 5-7 days to remove unreacted MA. After completing the dialysis process, the solution was transferred to the 50 mL centrifuge tubes, placed at −80° C. overnight, and freeze-dried (Free zone, 2.5 L, Labconco, USA) for 7 days to form a white GelMA aerogel. The freeze-dried GelMA were placed in moist-free condition at room temperature until use.

Fabrication of conventional (brittle) gelatin methacryloyl aerogel: Conventional brittle GelMA aerogel (BGA) was fabricated by dissolving freeze-dried GelMA (6% w/v) in DPBS at ~60° C. for ~20 min. Equal volumes of APS (1% w/v in DPBS) and 0.25% v/v TEMED were added to the GelMA solution to yield the GelMA pre-polymer. The pre-polymer mixture was transferred to a PDMS mold (6 mm×5 mm×2 mm) and allowed to crosslink for ~1 h at room temperature to form GelMA hydrogels with a final concentration of 3% w/v. The hydrogels were frozen at −80° C. for 24 h, followed by freeze-drying for 1 day to form 2 mm-thick brittle aerogels (BGAs). The samples were stored in a dry cabinet at room temperature before use in the experiments.

Fabrication of flexible aerogel: To fabricate FGAs, freeze-dried GelMA (6% w/v) was allowed to dissolve completely in DPBS at 60° C. for ~20 min. Then, an equal volume of refrigerated-cold APS (1% w/v in DPBS) was added to the GelMA solution as an initiator to adjust the final concentrations of GelMA and APS to 3% and 0.5% w/v, respectively. Next, TEMED (0.25% v/v) as a catalyst was added to the mixture and snapped vortexed for 3 s. The solution was immediately poured into pre-cooled PDMS molds (6 mm×5 mm×2 mm) and quickly transferred to liquid nitrogen to slow down the gelation process and form small and uniform interconnected ice crystals. The liquid nitrogen-frozen samples were placed at −80° C. for 24 h for slow completion of the crosslinking process. Finally, the samples were freeze-dried for 1 day to form an aerogel. The aerogels were placed in a tightly closed container and stored in a moist-free environment (i.e., dry cabinet) at ambient temperature until use.

Fabrication of the flexible aerogel-based electronic skin: The screen-printing was carried out with customized-built stainless steel stencils with 8 inch×10 inch dimensions, which were designed using AutoCAD software (Autodesk) and fabricated by Metal Etch Services (San Marcos, CA). The sensors were then fabricated via stencil masks to pattern two sets of iontophoresis (IP) electrodes, two reference electrodes, and other physiological electrodes. All the sensors and interconnections were printed with flexible Ag/AgCl inks. The electrochemical sensors were additionally patterned with carbon working and counter electrodes. After each printing step, the resulting device was baked at 60° C. in a convection oven for ca. 30 min to cure the conductive ink thoroughly. The biosensor electrodes were selectively modified by drop-casting their respective enzyme and polymer layers.

Lactate biosensor: Chitosan solution was mixed with LOx (40 mg/mL) in BSA solution at a ratio of 1:1 (v/v), followed by drop-casting the mixture (4 μL) onto the working electrode surface. All BSA solutions were prepared in PBS with a concentration of 10 mg/mL. The chitosan solution was prepared by dissolving in 0.1 M acetic acid with a concentration of 0.5 wt %. The PBS used in the electrode modifications for all the biosensors was prepared in 0.1 M with a pH of 7.4. The chitosan solution at concentration of 0.5 wt % was prepared by dissolving in 0.1 M acetic acid.

Alcohol biosensor: AOx (10-40 units/mg in BSA solution) was mixed with the chitosan solution at a ratio of 8:1 (v/v), and 4 μL of the mixture was dropped cast on to the working electrode surface.

Glucose biosensor: Equal volume of GOx (40 mg/mL) in BSA solution was mixed with the chitosan solution, and 4 μL of the mixture was dropped cast onto the working electrode surface. After drying at room temperature for 1 h, another 2 μL of the chitosan solution was dropped cast to all previously enzyme-modified surfaces, followed by storing at 4° C. overnight to ensure complete drying before the test.

For the dual IP process, epoxy molds of 1.5 mm thickness were printed via MONO resin 3D printer (Boston, MA). The cathode hydrogel solution was prepared by dissolving 4% w/v agarose in 0.1 M PBS solution at 150° C. while stirring until all agarose was visibly dissolved. Under the same temperature and stirring conditions, the anode hydrogel solution was prepared by dissolving a 4% w/v agarose solution in DI water. For the anode hydrogel, the temperature was immediately decreased to 60° C. after agarose dissolution, and pilocarpine was added at 2% w/v under continuous stirring. Either 200 μL (for the anode) or 300 μL (for the cathode) aliquots of the warm solutions were added into each circular mold and allowed them to solidify. After the solution cooled down and completely solidified in the mold, the gels were removed and stored in a wet chamber at 4° C. until further use.

In vitro calibration of the sensors: The printed sensors (glucose, lactate, alcohol biosensors, temperature, skin hydration, and ECG sensors) were calibrated separately in in vitro settings. The biosensors were calibrated using 0.1 M PBS (pH 7.4) and by spiking the corresponding analytes while recording the amperometric responses for glucose, lactate, and alcohol. Note that all amperometric responses were recorded after 1 min incubation in the sample solutions by applying a potential of −0.2 V (vs. Ag/AgCl) over 1 min. The selectivity of all sensors was evaluated by comparing their response to the target in the presence of common electroactive interferences, specifically 10 μM UA and 10 μM AA, in addition to the other biomarkers of interest (10 mM lactate and 1 mM glucose). Finally, one final standard addition of the target biomarker was added to verify that the sensors could accurately track concentration changes in such complex environments.

Glucose sensor: The calibration curve of the glucose sensor was obtained using an initial 100 μL PBS drop on the sensor surface. The solution was spiked with 4 μL of 0.1 M glucose solution to increase the concentration of glucose incrementally from 0 to 9 mM after each spiking. The selectivity of the glucose sensor was evaluated by performing amperometry while spiking the PBS successively with glucose. Glucose sensor was examined by performing 10 repetitive measurements of 2 mM glucose and calculating its relative response changes in %.

Alcohol sensor: The calibration curve of the alcohol sensor was obtained using an initial 100 μL PBS droplet on the sensor surface. The solution was spiked with 1 μL of 0.8 M ethanol solution to increase the concentration of alcohol incrementally from 0 to 100 mM after each spiking. The selectivity of the alcohol sensor was evaluated by performing CA while spiking the PBS measured successively with ethanol (20 mM), lactate (10 mM), glucose (0.2 mM), and ascorbic acid (10 μM).

Lactate sensor: The calibration curve of the lactate sensor was obtained using an initial PBS droplet with 100 μL volume on the sensor surface. The solution was spiked with 1 μL of 0.5 M lactate solution to increase the lactate concentration incrementally from 0 to 30 mM with CA at −0.2 V for 60 s after each spiking.

Temperature sensor: The calibration curve of the temperature sensor was characterized on a hot plate with gradually increasing temperature from 24 to 64° C. A thermocouple thermometer (Fluke 80PK-1) was used to obtain the local temperature on the temperature sensor, and a precision source meter (B2902A, Keysight) was used to monitor the resistance of the sensor by four-probe measurements.

Impedance sensor: The calibration curve of the impedance sensor was obtained over the 0-100 kHz range using a gelatin-based skin-mimicking phantom. Briefly, a 1:1 mixture of kerosene and safflower oil was brought to 70° C. Meanwhile, 4 g of gelatin was added to 21.5 mL of DI water and sonicated at a temperature of 80° C. for 15 min. 1.5 mL of propanol was then added to eliminate leftover bubbles. The oil mixture and gelatin solution were then mixed at a temperature of 46° C. and combined with 1.5 mL of Triton-X-100. The mixture was then poured, covered, and allowed to cool at room temperature until testing. To drive water evaporation and mimic different hydration states, the gel was placed on a hotplate at 45° C. in between measurements and allowed to cool completely before repeating measurements.

On-body validation of flexible aerogel-based electronic skin: Epidermal evaluation of the FGA-based E-skin was performed on healthy adult individuals with no heart conditions, diabetes, or chronic pain and in strict compliance with the protocol approved by the institutional review board at the University of California, Los Angeles (IRB #17-000170). Further, informed written consent of all participants was also obtained.

The FGA-based E-skin was placed on the forearm for all on-body validations. Volunteers were instructed to clean their forearm with soap and water and thoroughly dry it before the sensor was placed. An additional sensor fabrication step for on-body tests involved laser cutting double-sided tape (3M, United States) in providing better adhesion to the body and to form fluid chambers for the sensors. The glucose, lactate, alcohol, temperature, skin hydration, and ECG signals were validated with a commercial glucometer (CareTouch) (Brooklyn, NY), blood-lactate meter (the EDGE, USA), infrared thermometer (ETEKCITY) (Anaheim, CA), skin analyzer (SK-8, FANTEXY) (Shenzhen, China), and ECG monitor (Prince 180B, Heal Force) (Shanghai, China), respectively, before every set of measurements using the integrated sensor. Sweat stimulation and ISF extraction were realized simultaneously using an Agilent B2902A source-measure unit (Santa Clara, CA) to apply a current density of 0.3 mA cm² between the cathode and anode IP electrodes for 10 min. In all cases, a pre-conditioning step was carried out on the skin by first applying the same current density using agarose gels for 10 min, followed by immediate placement of the enzyme-modified FGA-based E-skin device with the anode pilocarpine gel on the conditioned area. All amperometric measurements were read at −0.2 V for 60 s immediately after 10 min of iontophoresis at 0.3 mA cm² with the pilocarpine-loaded gel. For all measurements, a single device was used for each volunteer to perform the “before” and “after” tests. The device was kept on the forearm of the volunteer throughout the entire experiment unless otherwise specified.

Exercise: Volunteers were instructed to mount an exercise bike for 25 min, followed by a 5 min cool-down period. Herein, the FGA-based E-skin was applied immediately before exercise to acquire the baseline values and subsequently removed and placed in a humid chamber so that exercise-induced sweat would not confound the glucose ISF measurements. Glucose and lactate levels, temperature, skin hydration, and ECG signals were acquired for the healthy volunteers before and after exercise. The FGA-based E-skin was removed from their skin during the 30 min workout on a stationary bike and kept for further use.

Alcohol intake: Alcohol levels were measured before and 20 min after alcohol consumption (250 mL red wine, alcohol 17% vol). The FGA-based E-skin was kept on the forearm of the volunteers during the entire experiment.

Food intake: Signals for ISF glucose were acquired for the healthy volunteers in the fasting state (12 h) and 20 min after consuming sugar-rich food. The FGA-based E-skin was kept on the forearm of the volunteer throughout the entire experiment.

Continuous on-body validation during exercise: The FGA-based E-skin was further tested by monitoring dynamic changes in sweat lactate during continuous physical activity. Individuals were asked to perform 30-min cycling activities at constant intensity activity. The skin temperature, skin hydration, ECG, and lactate levels were measured just before the start of the exercise, and the sweat was first generated. These signals were recorded within approximately 20 min of the exercise. These selected signals were also recorded on completion of the exercise for validation. Note that no IP electrodes/gels were needed for this protocol since the exercise-induced sufficient sweating.

Characterization and measurements: The air permeability was measured by using a customized setup based on the ASTM E96 standard; the testing process is as follows: a 20 mL sized glass bottle was filled with 15 mL of the distilled water, then sealed with a sample using double-sided tape. The bottle was placed in a chamber with a temperature of 35° C. and relative humidity of 40%. The mass of the bottle was measured every two days. The water vapor transmission was calculated based on the mass change. Electrical characterization was performed at room temperature using a CHI660E potentiostat (Englewood, CO) with its accompanying software. Temperature analysis was performed with an Agilent B2902A source-meter unit under an applied bias of 100 mV controlled by Keysight quick IV measurement curve software. Spectral reflectance and transmittance of the flexible GelMA aerogel in the sunlight and mid-IR range were obtained using a UV/Vis spectrophotometer (Shimadzu UV-3101 PC; Kyoto, Japan) with an integrating sphere and a Fourier transform IR spectrophotometer (Agilent 620/660; Santa Clara, CA), respectively.

Imaging with the scanning electron microscope (SEM): The surface morphology and porous interior structure of the FGA-based E-skin were characterized by a field-emission SEM (Supra 40 VP, Zeiss, Germany). Specifically, the GelMA aerogels (i.e., flexible and brittle) were adhered to double-sided carbon tape and placed on SEM holders. The samples' surfaces were coated with iridium using a sputter coater (South Bay Technology, USA). The images were captured at an accelerating voltage of 12 kV. ImageJ software (Version 1.52e, USA) was used to analyze pore size with a two-dimensional (2D) aspect.

Adhesion test: A standard wound closure test was conducted to assess the adhesive strength of FGA to porcine skin following the ASTM F2458-05 protocol with slight modifications. The porcine skin was purchased from a slaughterhouse and cut into pieces with dimensions of 20 mm×10 mm×2 mm. The skins were utilized in the experiments within 6 h of sacrificing the animals. Before the experiment, skin moisture was maintained by a wet towel in a humidifying chamber. Two pieces of the cut skin were put together such that their ends touched. The FGA (20 mm×5 mm×2 mm) was placed on top of the touching edges, and the adhesion test was performed immediately (0 h) or 8 h and 72 h after incubating the FGA on top of the skin in a humidifier chamber. Force-displacement curves were plotted using Bluehill software (Version 3, USA). Adhesive strength was calculated by dividing the force at the point of delamination by the cross-sectional area of the FGA under the force.

Assessment of pore interconnectivity: To analyze the interconnectivity of the aerogel's pores, dry samples were immersed in DPBS for 30 min to hydrate fully and to reach equilibrium liquid uptake. The weight of the hydrated samples was recorded. Next, the excess DPBS was lightly blotted with a Kimwipe for ~30 s, and the samples were weighed again. The interconnected pore volume was measured as the weight of the blotted DPBS divided by the total weight of the hydrated samples.

Rheological characterization: The rheological properties of flexible and brittle aerogels were characterized by a rheometer (Anton Paar, MCR 302, USA) using a sandblasted parallel-plate geometry (8 mm) with a gap of 1 mm at 25° C. Oscillatory strain sweep experiments were conducted over a range of 1-1000% strain at 1 Hz. The storage and loss moduli were plotted versus strain by Anton Paar Rheocompass software.

Contact angle characterization: The surface energy of the FGA-based E-skin was characterized by measuring the water contact angles at room temperature after depositing 20 μL of Milli-Q water on the flat surface of the aerogels and subsequent imaging. Images were analyzed by image-J software (version 1.53a, National Institute of Health, USA), and the water contact angle was reported as an average of three measurements for each sample.

Measurement of pore volume and surface area: The aerogels' pore volume and surface area were calculated by nitrogen adsorption-desorption isotherms using a volumetric instrument (Quadrasorb by Quantachrome) (Graz, Austria) at 77 K. The samples were degassed at 150° C. for 24 h in a vacuum before measurements. The surface area was measured in a range of 0.007<P/P₀<0.035 using the Brunauer-Emme-Teller (BET) method.

Mechanical tests: Mechanical tests were performed using an Instron instrument (model 5943, USA) and a load cell of 100 N. For the compression test, circular-shaped samples (8 mm diameter, 6 mm height) were soaked in DPBS for 30 min to fully hydrate. The hydrated samples were compressed at a constant rate of 2 mm/min. Young's modulus was calculated from the slope of the first 15% of the strain-stress curve for n=5 samples. The strain at which the material failed was considered a compressive failure strain. The maximum stress at which the material fails is considered compressive fracture stress. For tensile tests, rectangular shaped (20 mm×5 mm×2 mm) hydrated samples were glued on the paper from the top and bottom corners, and pulled away at a constant strain rate of 2 mm/min until failure. Stress-strain curves were recorded, and the slope of the linear range for the first 10% of the graphs was used to calculate the elastic modulus for five samples. The maximum stress before the abrupt drop in the graph was reported as tensile strength. The strain at which the stress suddenly dropped was recorded as tensile failure strain. Similarly, a cyclic tensile test was performed for 10 cycles when linearly stretched and relaxed by 50% to analyze the hysteresis behavior of the hydrated samples. In addition, a precision source meter (B2902A, Keysight) was used to monitor the resistance of the printed sensors every 10 cycles after 100 cyclic bending (bending curvature of 2 mm) and twisting) (180° deformations.

Degradation tests: The degradation behavior was monitored in the presence of 5 mL collagenase type II (2.5 U/mL in DPBS) at 37° C. The initial dry weight and dry weight of the degraded samples at each time point were measured after freeze-drying. Weight loss of the aerogels was calculated using the following equation:

$$Weight loss ( \% ) = ( {W}_{d} - {W}_{f} ) \times 100 / {W}_{d}$$

Where W_{d} is the initial dry sample weight, and W_{f} is the freeze-dried weight of the degraded aerogels at each time point. The weight loss was calculated for n=4 replicates.

Water uptake measurements: The ability to absorb water was tested by immersing the aerogels (8 mm diameter, 6 mm height) in DPBS and incubating them at 37° C. The initial dry weight and hydrated weight of the samples at different time points (2, 10, 30, 60, 120 and 720 min) after blotting the extra water were recorded. The water uptake was calculated using the below equation:

$$Water uptake ( \% ) = ( W h - Wd ) / Wd \times 100$$

where Wh is hydrated weight at each time point and Wd is the initial dry weight of the aerogels. The data are reported as mean±standard deviation (SD) of n=3 replicates. Dimension of each sample was also measured at the dry state and hydrated state to find any potential swelling upon water uptake.

Air permeability measurements: A commercial test chamber consisting of a feed and permeate chamber was constructed to measure the transient air permeation through the different sample films. Before loading, the film was mounted on a 47-mm nonporous brass sample disc with a hole (6.5 or 10 mm in diameter) in the center. The film covered the hole, and the edges were sealed by epoxy resin (Gorilla). The film was then carefully placed on a round filter paper with a diameter of 47 mm. The filter paper acted as a support with mechanical stability while having negligible resistance to the air. After sample loading, the chamber was fully degassed with a mechanical pump for over 4 h to a base pressure of 10⁻⁴ kPa.

The flux of the leak (dP_{d}/dt, leak) was tested before introducing the air to the chamber. After that, the air was introduced to the chamber, and the flux of downstream air (dP_{d}/dt, air) and upstream pressure of the air (Pᵤₚ) were recorded. With known downstream volume (V_{d}), gas constant R, measured sample thickness (1) and surface area (A), and temperature (T), the permeability is calculated by the equation:

$$Permeability = \frac{{V}_{d} l}{{P}_{u p} A R T} [ {( \frac{d {P}_{d}}{d t} )}_{g a s} - {( \frac{d {P}_{d}}{d t} )}_{l e a k} ]$$

Where V_{d} is the downstream volume, 1 is the sample thickness, A is the surface area, T is the temperature, Pᵤₚ is the upstream pressure of the air, P_{d} is the downstream pressure, and R is the gas constant.

Moisture permeability measurements: The moisture permeability of each film was measured on a customized-built test system based on the ASTM E96 standard. The testing process is summarized below: a 20 mL plastic bottle was filled with 17 mL of distilled water, then sealed with a sample using double-sided tape. The bottle was placed in a chamber with a constant temperature of 36° C. and relative humidity of 45%. The mass of the bottle was measured every 24 h. The moisture permeability performance was calculated based on the mass change.

Cell viability assay: Human dermal fibroblasts were cultured in DMEM supplemented with 10% FBS and 1% Pen-Strep until ~90% confluency. The confluent cells were trypsinized and seeded either directly on an FGA-based E-skin (10 mm×10 mm×1 mm) or in a 12-well plate at a density of 5000 cells/well for the direct and indirect cell viability assessments, respectively. For the indirect method, 24 h after cell seeding, the FGA-based E-skin (10 mm×10 mm×1 mm) was placed in the transwells (4 μm mesh) on the upper compartments of the wells and kept in a 5% CO₂ incubator (ThermoFisher Scientific, United States). PrestoBlue cell viability assay in a ratio of (1:9 in DMEM) was conducted according to the manufacturer's instructions on days 1, 3, and 7 of the experiment. Fluorescence intensity corresponding to the metabolic activity of the cells was measured after 1.5 h incubation of the cells with PrestoBlue using a microplate reader (VARIOSKAN LUX, Thermo Scientific, United States) at excitation/emission of 530/590 nm. A live/dead assay was performed on day 7 of incubation after staining the cells with calcein-AM (0.5 μL) and ethidium homodimer-1 (2 μL) in DPBS (1 mL). After 15 min incubation, the staining solution was discarded, and the cells were washed twice with DPBS. Fluorescence images were captured by Keyence fluorescence microscope (BZ-X700 Series) using red and green channels for ethidium homodimer-1 and calcein-AM, respectively.

Statistical analysis: Quantitative data were statistically analyzed using GraphPad Prism software (version 9.3.0). One-way analysis of variance (ANOVA) followed by Tukey's multiple comparison test or Two-way ANOVA test were applied, where appropriate. Statistically significant differences were presented as * (p<0.05), ** (p<0.01), *** (p<0.001), and **** (p<0.0001). Statistically non-significant is shown by ns.

### EQUIVALENTS AND SCOPE

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, aerogels, materials, kits, and/or methods, if such features, systems, aerogels, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite aerogels “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a crosslinked polymer aerogel comprising gelatin or a derivative thereof, the aerogel having an average pore diameter of less than 50 micrometers, an average pore interconnectivity of at least 65%, and an average moisture permeability of at least 2500 g/(m² day).

2. The article of claim 1 wherein the gelatin or a derivative thereof comprises crosslinked reactive groups.

3. The article of claim 2, wherein the reactive groups comprise an allyl group.

4. The article of any one of claim 2 or 3, wherein the reactive groups comprise a vinyl group.

5. The article of any one of claims 1-4, wherein the gelatin or a derivative thereof comprises methacryloyl gelatin.

6. The article of any one of claims 1-5, further comprising an adhesive in contact with a first surface of the aerogel.

7. The article of claim 6, wherein the adhesive comprises double sided tape.

8. The article of any one of claims 1-7, further comprising an electronic element in contact with the aerogel.

9. The article of claim 8, wherein the electronic element comprises a sensor.

10. The article of claim 9, wherein the sensor is an electrochemical sensor.

11. The article of any one of claim 9 or 10, wherein the sensor is a biopotential sensor.

12. The article of any one of claims 9-11, wherein the sensor is a temperature sensor.

13. The article of any one of claims 9-12, wherein the sensor is an impedance sensor.

14. The article of any one of claims 1-13, wherein the aerogel has an average elastic modulus of between 3 kPa and 6 kPa.

15. The article of any one of claims 1-14, wherein the aerogel has an average air permeability of at least 1.9×10⁷ barrer.

16. The article of any one of claims 1-15, wherein the aerogel has an average degree of hysteresis of less than 10% when linearly stretched and relaxed by 50% for at least 100 cycles.

17. The article of any one of claims 1-16, wherein the aerogel is able to absorb a mean weight of a fluid equal to between 500% w/w and 1000% w/w of the aerogels dry weight.

18. The article of claim 17, wherein absorbing the fluid causes the aerogel to swell by less than 1% v/v.

19. The article of any one of claims 1-18, wherein the aerogel has an average mechanical bending deformation of at least 35 Pa under less than 15% strain.

20. The article of any one of claims 1-19, wherein the aerogel has an average adhesive strength of between 1 kPa and 4 kPa as determined by an adhesion test.

21. The article of any one of claims 1-20, wherein the aerogel has an average moisture content of less than 1%.

22. An article, comprising:
a crosslinked polymer aerogel comprising gelatin or a derivative thereof, the aerogel having an average pore diameter of less than 50 micrometers, an average pore interconnectivity of more than 65%, and an average moisture content of less than 1%.

23. An article, comprising:
a crosslinked polymer aerogel comprising gelatin or a derivative thereof, wherein the aerogel has an average elastic modulus of between 3 kPa and 6 kPa.

24. The article of claim 23, wherein the gelatin or derivative thereof comprises crosslinked reactive groups.

25. The article of claim 24, wherein the reactive groups comprise an allyl group.

26. The article of claim 24 or 25, wherein the reactive groups comprise a vinyl group.

27. The article of any one of claims 24-26, wherein the gelatin or derivative thereof comprises methacryloyl gelatin.

28. The article of any one of claims 23-27, further comprising an adhesive in contact with the aerogel.

29. The article of claim 28, wherein the adhesive comprises double sided tape

30. The article of any one of claims 23-29, further comprising an electronic element in contact with the aerogel.

31. The article of claim 30, wherein the electronic element comprises a sensor.

32. The article of claim 31, wherein the sensor is an electrochemical sensor.

33. The article of any one of claim 31 or 32, wherein the sensor is a biopotential sensor.

34. The article of any one of claims 31-33, wherein the sensor comprises a temperature sensor.

35. The article of any one of claims 31-34, wherein the sensor comprises an impedance sensor.

36. The article of any one of claims 23-35, wherein the aerogel has an average pore diameter of less than 50 micrometers.

37. The article of any one of claims 23-36, wherein the aerogel has an average pore interconnectivity of at least 65%.

38. The article of any one of claims 23-37, wherein the aerogel has an average moisture permeability of at least 2500 g/(m² day).

39. The article of any one of claims 23-38, wherein the aerogel has an average air permeability of at least 1.9×10⁷ barrer.

40. The article of any one of claims 23-39, wherein the aerogel has an average degree of hysteresis of less than 10% when linearly stretched and relaxed by 50% for at least 100 cycles.

41. The article of any one of claims 23-40, wherein the aerogel absorbs a mean weight of fluid equal to between 500% w/w and 1000% w/w of the aerogels dry weight.

42. The article of claim 41, wherein absorbing the fluid causes the aerogel to swell by less than 1% v/v.

43. The article of any one of claims 23-42, wherein the aerogel has an average mechanical bending deformation of at least 35 Pa under less than 15% strain.

44. The article of any one of claims 23-43, wherein the aerogel has an average adhesive strength of between 1 kPa and 4 kPa as determined by an adhesion test.

45. The article of any one of claims 23-44, wherein the aerogel has an average moisture content of less than 1%.

46. An article, comprising:
a crosslinked polymer aerogel comprising gelatin or a derivative thereof, the aerogel having an average pore diameter of less than 50 micrometers, an average pore interconnectivity of more than 65%, and an average moisture permeability such that the aerogel swells by less than 1 vol % after absorbing between 500% w/w and 1000% w/w of saline.

47. An article, comprising:
a crosslinked polymer aerogel comprising gelatin or a derivative thereof, the aerogel having an average pore diameter of less than 50 micrometers, an average pore interconnectivity of more than 65%, and an average degree of hysteresis of less than 10% when linearly stretched and relaxed by 50% for at least 100 cycles.

48. A method, comprising:
crosslinking a polymer comprising gelatin or a derivative thereof in a solution to form a crosslinked solution;
causing the crosslinked solution to gel; and
freeze-drying the gel to form a crosslinked polymer aerogel.

49. The method of claim 48, wherein the gelatin or derivative thereof comprises a plurality of reactive groups.

50. The method of claim 49, wherein the reactive groups comprise an allyl group.

51. The method of any one of claim 49 or 50, wherein the reactive groups comprise a vinyl group.

52. The method of any one of claims 49-51, wherein the gelatin or derivative thereof comprises methacryloyl gelatin.

53. The method of any one of claims 48-52, wherein the solution further comprises a catalyst.

54. The method of claim 53, wherein the catalyst comprises TEMED.

55. The method of any one of claims 48-53, wherein the solution further comprises an initiator.

56. The method of claim 55, wherein the initiator comprises ammonium persulfate (APS).

57. The method of any one of claims 48-56, further comprising cooling the solution to a temperature less than or equal to −196° C.

58. The method of any one of claims 48-57, further comprising cooling the solution to a temperature less than or equal to −80° C.

59. The method of claim 57 or 58, wherein cooling the solution causes the crosslinked solution to gel in less than or equal to 24 hours to produce a cryogel.

60. The method of claim 59, wherein the cryogel comprises a network of interconnected ice crystals within the cryogel.

61. The method of any one of claims 48-60, wherein freeze-drying the gel removes the network of interconnected ice crystals within the cryogel to produce the crosslinked polymer aerogel.

62. A method comprising adhering any one of the articles of claims 1-61 to a dermal surface of a subject.

63. The method of claim 62, wherein adhering any one of the articles does not require the use of an adhesive.

64. The method of claim 62, wherein adhering any one of the articles does require the use of an adhesive.
